SEQUENCE LISTING (74 sequences)

SEQ ID NO: 1 - protein, 18 aa, Homo sapiens
FSISRGIYSCIHGVHIEE

SEQ ID NO: 2 - protein, 18 aa, Artificial sequence
  GluN2A motif mutated with 2 Ile residues mutated to Ala residues
FSISRGAYSCAHGVHIEE

SEQ ID NO: 3 - protein, 18 aa, Artificial sequence
  GluN2A core motif mutated with 3 Ile residues mutated to Ala residues
FSASRGAYSCAHGVHIEE

SEQ ID NO: 4 - protein, 18 aa, Homo sapiens
FSISRGMYSCIHGVHIEE

SEQ ID NO: 5 - protein, 18 aa, Homo sapiens
FSISRGIYRCIHGVHIEE

SEQ ID NO: 6 - protein, 18 aa, artificial sequence
  Homo sapiens
FSISRGIYSCIHGVHNEE

SEQ ID NO: 7 - protein, 18 aa, Homo sapiens
FSISRGIYSCIHGVAIEE

SEQ ID NO: 8 - protein, 18 aa, Homo sapiens
FSISRGIYSCMHGVAIEE

SEQ ID NO: 9 - protein, 33 aa, homo sapiens
TGVCSDRPGLLFSISRGIYSCIHGVHIEEKKKS

SEQ ID NO: 10 - protein, 33 aa, Artificial sequence
  GluN2A core motif mutated with 2 Ile residues mutated to Ala residues plus flanking regions
TGVCSDRPGLLFSISRGAYSCAHGVHIEEKKKS

SEQ ID NO: 11 - protein, 33 aa, Artificial sequence
  GluN2A core motif mutated with 3 Ile residues mutated to Ala residues plus flanking regions
TGVCSDRPGLLFSASRGAYSCAHGVHIEEKKKS

SEQ ID NO: 12 - protein, 33 aa, Homo sapiens
TGVCSDRPGLLFSISRGMYSCIHGVHIEEKKKS

SEQ ID NO: 13 - protein, 33 aa, Homo sapiens
TGVCSDRPGLLFSISRGIYRCIHGVHIEEKKKS

SEQ ID NO: 14 - protein, 33 aa, Homo sapiens
TGVCSDRPGLLFSISRGIYSCIHGVHNEEKKKS

SEQ ID NO: 15 - protein, 33 aa, Homo sapiens
MGVCSGKPGMVFSISRGIYSCIHGVAIEERQSV

SEQ ID NO: 16 - protein, 33 aa, Homo sapiens
MGVCSGKPGMVFSISRGIYSCMHGVAIEERQSV

SEQ ID NO: 17 - protein, 29 aa, Unknown
  Glycosylphosphatidylinositol (GPI) signal anchor sequence
LENGGTSLSEKTVLLLVTPFLAAAWSLHP

SEQ ID NO: 18 - protein, 18 aa, Unknown
  C-terminal targeting signal of K-Ras4B
SKDGKKKKKKSRTRCTVM

SEQ ID NO: 19 - protein, 9 aa, Human immunodeficiency virus
RKKRRQRRR

SEQ ID NO: 20 - protein, 9 aa, Unknown
  HA tag
YPYDVPDYA

SEQ ID NO: 21 - protein, 18 aa, Artificial sequence
  GluN2A motif mutated with 2 Ile residues and one His residue mutated to Ala residues
FSISRGAYSCAHGVAIEE

SEQ ID NO: 22 - protein, 18 aa, Homo sapiens
FSISRGIYSCIRGVAIEE

SEQ ID NO: 23 - protein, 15 aa, Artificial sequence
  GluN2A deletion mutant fragment exhibiting only flanking regions
TGVCSDRPGLLKKKS

SEQ ID NO: 24 - protein, 33 aa, Artificial sequence
  GluN2A motif mutated with 2 Ile residues and one His residue mutated to Ala residues plus flanking regions
TGVCSDRPGLLFSISRGAYSCAHGVAIEEKKKS

SEQ ID NO: 25 - protein, 15 aa, Artificial sequence
  GluN2B deletion mutant fragment exhibiting only flanking regions
MGVCSGKPGMVRQSV

SEQ ID NO: 26 - protein, 33 aa, Artificial sequence
  GluN2B motif mutated with 2 Ile residues mutated to Ala residues plus flanking regions
MGVCSGKPGMVFSISRGAYSCAHGVAIEERQSV

SEQ ID NO: 27 - protein, 33 aa, Artificial sequence
  GluN2B motif mutated with 2 Ile residues mutated to Ala residues and one Ala residue mutated to His plus flanking regions
MGVCSGKPGMVFSISRGAYSCAHGVHIEERQSV

SEQ ID NO: 28 - protein, 33 aa, Homo sapiens
MGVCSGKPGMVFSISRGIYSCIRGVAIEERQSV

SEQ ID NO: 29 - DNA, 20 bp, Homo sapiens
gggatctgccacaacgagaa

SEQ ID NO: 30 - DNA, 20 bp, Homo sapiens
ctcccgtttcctttctgcct

SEQ ID NO: 31 - DNA, 23 bp, Homo sapiens
cctatgctttgcaacttgtctca

SEQ ID NO: 32 - DNA, 22 bp, Homo sapiens
ttggatgaagtcagcagctctt

SEQ ID NO: 33 - DNA, 18 bp, Homo sapiens
gcgtattctacatgctgg

SEQ ID NO: 34 - DNA, 18 bp, Homo sapiens
cagggctcctgcaagaag

SEQ ID NO: 35 - DNA, 20 bp, Homo sapiens
gaagctctctggctcactgg

SEQ ID NO: 36 - protein, 20 aa, Homo sapiens
AACTGCCCAAATCCCACACA

SEQ ID NO: 37 - DNA, 20 bp, Homo sapiens
acctcagctcaccacatgac

SEQ ID NO: 38 - protein, 20 aa, Homo sapiens
GATGACTCCCGTCGGATGAA

SEQ ID NO: 39 - DNA, 19 bp, Homo sapiens
cacaatgagaagaatgagg

SEQ ID NO: 40 - DNA, 19 bp, Homo sapiens
ggtataaattaggcaaact

SEQ ID NO: 41 - protein, 1446 aa, Artificial sequence
  GluN2A LACK SEQ ID 1, Q00959, NMDE1_RAT
MGRLGYWTLLVLPALLVWRDPAQNAAAEKGPPALNIAVLLGHSHDVTERELRNLWGPEQATGLPLDVNVVALLMNRTDPKSLITHVCDLMSGARIHGLVFGDDTDQEAVAQMLDFISSQTFIPILGIHGGASMIMADKDPTSTFFQFGASIQQQATVMLKIMQDYDWHVFSLVTTIFPGYRDFISFIKTTVDNSFVGWDMQNVITLDTSFEDAKTQVQLKKIHSSVILLYCSKDEAVLILSEARSLGLTGYDFFWIVPSLVSGNTELIPKEFPSGLISVSYDDWDYSLEARVRDGLGILTTAASSMLEKFSYIPEAKASCYGQAEKPETPLHTLHQFMVNVTWDGKDLSFTEEGYQVHPRLVVIVLNKDREWEKVGKWENQTLSLRHAVWPRYKSFSDCEPDDNHLSIVTLEEAPFVIVEDIDPLTETCVRNTVPCRKFVKINNSTNEGMNVKKCCKGFCIDILKKLSRTVKFTYDLYLVTNGKHGKKVNNVWNGMIGEVVYQRAVMAVGSLTINEERSEVVDFSVPFVETGISVMVSRSNGTVSPSAFLEPFSASVWVMMFVMLLIVSAIAVFVFEYFSPVGYNRNLAKGKAPHGPSFTIGKAIWLLWGLVFNNSVPVQNPKGTTSKIMVSVWAFFAVIFLASYTANLAAFMIQEEFVDQVTGLSDKKFQRPHDYSPPFRFGTVPNGSTERNIRNNYPYMHQYMTRFNQRGVEDALVSLKTGKLDAFIYDAAVLNYKAGRDEGCKLVTIGSGYIFASTGYGIALQKGSPWKRQIDLALLQFVGDGEMEELETLWLTGICHNEKNEVMSSQLDIDNMAGVFYMLAAAMALSLITFIWEHLFYWKLRFCFTGVCSDRPGLLKKKSPDFNLTGSQSNMLKLLRSAKNISNMSNMNSSRMDSPKRATDFIQRGSLIVDMVSDKGNLIYSDNRSFQGKDSIFGDNMNELQTFVANRHKDNLSNYVFQGQHPLTLNESNPNTVEVAVSTESKGNSRPRQLWKKSMESLRQDSLNQNPVSQRDEKTAENRTHSLKSPRYLPEEVAHSDISETSSRATCHREPDNNKNHKTKDNFKRSMASKYPKDCSDVDRTYMKTKASSPRDKIYTIDGEKEPSFHLDPPQFVENITLPENVGFPDTYQDHNENFRKGDSTLPMNRNPLHNEDGLPNNDQYKLYAKHFTLKDKGSPHSEGSDRYRQNSTHCRSCLSNLPTYSGHFTMRSPFKCDACLRMGNLYDIDEDQMLQETGNPATREEVYQQDWSQNNALQFQKNKLRINRQHSYDNILDKPREIDLSRPSRSISLKDRERLLEGNLYGSLFSVPSSKLLGNKSSLFPQGLEDSKRSKSLLPDHASDNPFLHTYGDDQRLVIGRCPSDPYKHSLPSQAVNDSYLRSSLRSTASYCSRDSRGHSDVYISEHVMPYAANKNTMYSTPRVLNSCSNRRVYKKMPSIESDV

SEQ ID NO: 42 - protein, 1464 aa, Artificial sequence
  GluN2B LACK SEQ ID 7, Q00960, NMDE2_RAT
MKPSAECCSPKFWLVLAVLAVSGSKARSQKSPPSIGIAVILVGTSDEVAIKDAHEKDDFHHLSVVPRVELVAMNETDPKSIITRICDLMSDRKIQGVVFADDTDQEAIAQILDFISAQTLTPILGIHGGSSMIMADKDESSMFFQFGPSIEQQASVMLNIMEEYDWYIFSIVTTYFPGYQDFVNKIRSTIENSFVGWELEEVLLLDMSLDDGDSKIQNQLKKLQSPIILLYCTKEEATYIFEVANSVGLTGYGYTWIVPSLVAGDTDTVPSEFPTGLISVSYDEWDYGLPARVRDGIAIITTAASDMLSEHSFIPEPKSSCYNTHEKRIYQSNMLNRYLINVTFEGRNLSFSEDGYQMHPKLVIILLNKERKWERVGKWKDKSLQMKYYVWPRMCPETEEQEDDHLSIVTLEEAPFVIVESVDPLSGTCMRNTVPCQKRIISENKTDEEPGYIKKCCKGFCIDILKKISKSVKFTYDLYLVTNGKHGKKINGTWNGMIGEVVMKRAYMAVGSLTINEERSEVVDFSVPFIETGISVMVSRSNGTVSPSAFLEPFSADVWVMMFVMLLIVSAVAVFVFEYFSPVGYNRCLADGREPGGPSFTIGKAIWLLWGLVFNNSVPVQNPKGTTSKIMVSVWAFFAVIFLASYTANLAAFMIQEEYVDQVSGLSDKKFQRPNDFSPPFRFGTVPNGSTERNIRNNYAEMHAYMGKFNQRGVDDALLSLKTGKLDAFIYDAAVLNYMAGRDEGCKLVTIGSGKVFASTGYGIAIQKDSGWKRQVDLAILQLFGDGEMEELEALWLTGICHNEKNEVMSSQLDIDNMAGVFYMLGAAMALSLITFICEHLFYWQFRHCFMGVCSGKPGMVRQSVMNSPTATMNNTHSNILRLLRTAKNMANLSGVNGSPQSALDFIRRESSVYDISEHRRSFTHSDCKSYNNPPCEENLFSDYISEVERTFGNLQLKDSNVYQDHYHHHHRPHSIGSTSSIDGLYDCDNPPFTTQPRSISKKPLDIGLPSSKHSQLSDLYGKFSFKSDRYSGHDDLIRSDVSDISTHTVTYGNIEGNAAKRRKQQYKDSLKKRPASAKSRREFDEIELAYRRRPPRSPDHKRYFRDKEGLRDFYLDQFRTKENSPHWEHVDLTDIYKERSDDFKRDSVSGGGPCTNRSHLKHGTGEKHGVVGGVPAPWEKNLTNVDWEDRSGGNFCRSCPSKLHNYSSTVAGQNSGRQACIRCEACKKAGNLYDISEDNSLQELDQPAAPVAVTSNASSTKYPQSPTNSKAQKKNRNKLRRQHSYDTFVDLQKEEAALAPRSVSLKDKGRFMDGSPYAHMFEMPAGESSFANKSSVPTAGHHHNNPGSGYMLSKSLYPDRVTQNPFIPTFGDDQCLLHGSKSYFFRQPTVAGASKTRPDFRALVTNKPVVVTLHGAVPGRFQKDICIGNQSNPCVPNNKNPRAFNGSSNGHVYEKLSSIESDV

SEQ ID NO: 43 - protein, 16 aa, Homo sapiens
TGVCSDRPGLLFSISR

SEQ ID NO: 44 - protein, 17 aa, Homo sapiens
GIYSCIHGVHIEEKKKS

SEQ ID NO: 45 - protein, 16 aa, Homo sapiens
MGVCSGKPGMVFSISR

SEQ ID NO: 46 - protein, 17 aa, Homo sapiens
GIYSCIHGVAIEERQSV

SEQ ID NO: 47 - DNA, 30 bp, Artificial sequence
  GRIN2AdeltaSEQ ID NO1 forward primer
ccgggctgctccacattgaagaaaagaaga

SEQ ID NO: 48 - DNA, 30 bp, Artificial sequence
  GRIN2AdeltaSEQ ID NO1 reverse primer
tcttcaatgtggagcagcccgggccggtca

SEQ ID NO: 49 - DNA, 22 bp, artificial sequence
  GRIN2AdeltaC9 forward primer
cgctaaagagtcctaggtatct

SEQ ID NO: 50 - DNA, 41 bp, Artificial sequence
  GRIN2AdeltaC9 reverse primer
gtctgctcgaagcggccgcttacttgtacactcgtctattg

SEQ ID NO: 51 - DNA, 36 bp, Artificial sequence
  GRIN2A-I2A2 forward primer
ctccatcagcaggggcgcctatagttgcgcccatgg

SEQ ID NO: 52 - DNA, 36 bp, Artificial sequence
  GRIN2A-I2A2 reverse primer
ccatgggcgcaactataggcgcccctgctgatggag

SEQ ID NO: 53 - DNA, 32 bp, Artificial sequence
  GRIN2A-I3A3 forward primer
ggctgctcttctccgccagcaggggcgcctat

SEQ ID NO: 54 - DNA, 32 bp, artificial sequence
  GRIN2A-I3A3 reverse primer
ataggcgcccctgctggcggagaagagcagcc

SEQ ID NO: 55 - DNA, 30 bp, Artificial sequence
  GRIN2A-I2A2HA forward primer
tgcgcccatggggtagccattgaagaaaag

SEQ ID NO: 56 - DNA, 30 bp, Artificial sequence
  GRIN2A-I2A2HA reverse primer
cttttcttcaatggctaccccatgggcgca

SEQ ID NO: 57 - DNA, 31 bp, Artificial sequence
  GRIN2A-I867M forward primer
catcagcaggggcatgtatagttgcatccat

SEQ ID NO: 58 - DNA, 31 bp, Artificial sequence
  GRIN2A-I867M reverse primer
atggatgcaactatacatgcccctgctgatg

SEQ ID NO: 59 - DNA, 23 bp, Artificial sequence
  GRIN2A-S869R forward primer
ggcatctatagatgcatccatgg

SEQ ID NO: 60 - DNA, 23 bp, Artificial sequence
  GRIN2A-S869R reverse primer
ccatggatgcatctatagatgcc

SEQ ID NO: 61 - DNA, 25 bp, Artificial sequence
  GRIN2A-I876N forward primer
catccatggggtacacaatgaagaa

SEQ ID NO: 62 - DNA, 25 bp, Artificial sequence
  GRIN2A-I876N reverse primer
ttcttcattgtgtaccccatggatg

SEQ ID NO: 63 - DNA, 27 bp, Artificial sequence
  GRIN2BdeltaSEQ ID NO7 forward primer
tggcatggtcgccatagaggagcgcca

SEQ ID NO: 64 - DNA, 29 bp, Artificial sequence
  GRIN2BdeltaSEQ ID NO7 reverse primer
cctctatggcgaccatgccaggcttgcca

SEQ ID NO: 65 - DNA, 23 bp, Artificial seqeunce
  GRIN2BdeltaC9 forward primer
gtggattgggaggaccggtctgg

SEQ ID NO: 66 - DNA, 33 bp, Artificial sequence
  GRIN2BdeltaC9 reverse primer
ggcaagaattctcactcataaacatgtccattg

SEQ ID NO: 67 - DNA, 36 bp, Artificial sequence
  GRIN2B-I2A2 forward primer
ctccatcagcagaggtgcctacagctgtgcccatgg

SEQ ID NO: 68 - DNA, 36 bp, Artificial sequence
  GRIN2B-I2A2 reverse primer
ccatgggcacagctgtaggcacctctgctgatggag

SEQ ID NO: 69 - DNA, 31 bp, Artificial sequence
  GRIN2B-I2A2AH forward primer
tgtgcccatggggtacacatagaggagcgcc

SEQ ID NO: 70 - DNA, 31 bp, Artificial sequence
  GRIN2B-I2A2AH reverse primer
ggcgctcctctatgtgtaccccatgggcaca

SEQ ID NO: 71 - DNA, 27 bp, Artificial sequence
  GRIN2B-I872M forward primer
atctacagctgtatgcatggggtagcc

SEQ ID NO: 72 - DNA, 27 bp, Artificial sequence
  GRIN2B-I872M reverse primer
ggctaccccatgcatacagctgtagat

SEQ ID NO: 73 - DNA, 30 bp, Artificial sequence
  GRIN2B-H873R forward primer
ctacagctgtatccgtggggtagccataga

SEQ ID NO: 74 - DNA, 30 bp, Artificial sequence
  GRIN2B-H873R reverse primer
tctatggctaccccacggatacagctgtag

(19) United States
(12) Patent Application Publication
(10) Pub. No.: US 2023/0340060 A1
(43) Pub. Date: Oct. 26, 2023

(54) NOVEL MEANS TO PREDICT AND MANIPULATE NMDA RECEPTOR-MEDIATED TOXICITY

(71) Applicant: FundaMental Pharma GmbH, Heidelberg (DE)

(72) Inventors: Hilmar BADING, Heidelberg (DE); Jing YAN, Heidelberg (DE)

(21) Appl. No.: 18/041,226

(22) PCT Filed: Aug. 12, 2021
(86) PCT No.: PCT/EP2021/072452

§ 371 Date: Feb. 10, 2023

(30) Foreign Application Priority Data
Aug. 12, 2020 (EP) 20190722.7

(51) Int. Cl. C07K 14/705; A61P 35/00; C07K 16/28; C12Q 1/6869

(52) U.S. Cl. CPC C07K 14/705; A61P 35/00; C07K 16/28; C12Q 1/6869; C07K 2319/10

(57) ABSTRACT

The present invention relates to the field of neurodegenerative processes. In particular, the present invention relates to a polypeptide comprising a sequence motif of the GluN2A and GluN2B protein, the motif having an amino acid sequence according to SEQ ID NO:1 or SEQ ID NO:7 or a variant sequence thereof, which is useful in studying and modulating NMDA receptor mediated toxicity. The present invention also relates to fusion proteins comprising said polypeptides, nucleic acids encoding the same, and respective (host) cells and compositions. The present invention also relates to compounds binding to said polypeptides, as well as methods for assessing the susceptibility of an individual to NMDA receptor mediated toxicity.

The present invention relates to the field of neurodegenerative processes. In particular, the present invention relates to a polypeptide comprising a sequence motif of the GluN2A and GluN2B protein, the motif having an amino acid sequence according to SEQ ID NO:1 or SEQ ID NO:7 or a variant sequence thereof, which is useful in studying and modulating NMDA receptor mediated toxicity. The present invention also relates to fusion proteins comprising said polypeptides, nucleic acids encoding the same, and respective (host) cells and compositions. The present invention also relates to compounds binding to said polypeptides, as well as methods for assessing the susceptibility of an individual to NMDA receptor mediated toxicity.

Neurodegenerative diseases are devastating diseases involving the progressive loss of the structure and function of neurons, eventually causing death of the neurons. Neurodegeneration may be acute or slowly progressive, but both types of neurodegeneration often involve increased death signaling by extrasynaptic NMDA receptors caused by elevated extracellular glutamate concentrations or relocalization of NMDA receptors to extrasynaptic sites. NMDA receptors are glutamate- and voltage-gated ion channels that are permeable for calcium. They can be categorized according to their subcellular location as synaptic and extrasynaptic NMDA receptors. The subunit composition of the receptors within and outside synaptic contacts is similar, although, in addition to carrying the common Glutamate Ionotropic Receptor NMDA Type Subunit 1 (GluN1) subunit, extrasynaptic NMDA receptors contain preferentially the GluN2B subunit, whereas GluN2A is the predominant subunit in synaptic NMDA receptors. The cellular consequences of synaptic versus extrasynaptic NMDA receptor stimulation are dramatically different. Synaptic NMDA receptors initiate physiological changes in the efficacy of synaptic transmission. They also trigger calcium signaling pathways to the cell nucleus that activate gene expression responses that are critical for the long-term implementation of virtually all behavioral adaptations. Most importantly, synaptic NMDA receptors, acting via nuclear calcium, are strong activators of neuronal structure-protective and survival-promoting genes. In striking contrast, extrasynaptic NMDA receptors trigger cell death pathways. Within minutes after extrasynaptic NMDA receptor activation, the mitochondrial membrane potential breaks down, followed by mitochondrial permeability transition. Extrasynaptic NMDA receptors also strongly antagonize excitation-transcription coupling and disrupt nuclear calcium-driven adaptogenomics because they trigger a cyclic adenosine monophosphate (cAMP)-responsive element-binding protein (CREB) shutoff pathway, inactivate extracellular signal-regulated kinase (ERK)-MAPK signaling, and lead to nuclear import of class IIa histone deacetylases (HDACs) and the pro-apoptotic transcription factor Foxo3A. This affects activity regulation of many genes, including brain-derived neurotrophic factor (Bdnf) and vascular endothelial growth factor D (Vegfd), that are vital for the maintenance of complex dendritic architecture and synaptic connectivity as well as the build-up of a neuroprotective shield. In addition, given the short reach of activated ERK1/2, their shut-off by extrasynaptic NMDA receptors disrupts important local signaling events including dendritic mRNA translation and AMPA (α-amino-3-hydroxy-5-methyl-4-isoxazolepropionic acid) receptor trafficking that controls the efficacy of synaptic transmission. Thus, extrasynaptic NMDA receptor signaling is characterized by the initiation of a pathological triad with mitochondrial dysfunction, deregulation of transcription, and loss of integrity of neuronal structures and connectivity.

However, and so far, the molecular basis of toxic signaling of NMDA receptors remained unknown. This is unfortunate because there is a need in the art for, e.g., diagnostic tools to determine the risk of developing or suffering from neurodegenerative processes related to NMDA receptor signaling. The problem to be solved by the present invention was thus to provide new means to study, predict, and/or modulate NMDA receptor-mediated toxicity.

This problem is solved by the subject-matter as set forth in the appended claims and in the description below.

The inventors of the present invention have surprisingly found that an evolutionary highly conserved stretch of 18 amino acids with four regularly spaced isoleucines located within the intracellular, near-membrane portion of GluN2A and GluN2B is responsible for NMDA receptor-mediated toxicity. Polypeptides with the corresponding sequence are sufficient to induce NMDA receptor-mediated toxicity, resulting in neuronal cell death. The inventors found in a mutational analysis that the isoleucines as well as certain amino acids flanking the isoleucines within the highly conserved stretch of 18 amino acids determine the toxicity of NMDA receptor channels in non-neuronal cells, including human embryonic kidney 293 (HEK293) cells. Moreover, they found variants of this polypeptide motif of the GluN2A/GluN2B protein, which are less or more toxic. Some of these variants correspond to Single Nucleotide Polymorphisms (SNPs) which are present in the gene pool of the human population. It is contemplated that there are human subjects, which due to this variation are more prone to NMDA receptor mediated toxicity, and thus to neurodegenerative processes, than others, bringing about implications for diagnosis and prognosis of such diseases. It is also contemplated that there are human subjects, which due to this variation are less prone to NMDA receptor mediated toxicity, and thus to neurodegenerative processes, than others, and thus have the ‘advantage’ of undergoing no or less neuronal death in condition that are harmful for neurons. These conditions include hypoxic ischemic conditions, traumatic brain injury, glaucoma, cerebral microangiopathies, neuropathies due to metabolic diseases such as diabetes, and genetic or non-genetic pre-dispositions to neurodegenerative diseases including Alzheimer's disease, Parkinson's disease, Huntington's disease, Amyotrophic Lateral Sclerosis (ALS).

Therefore, the present invention relates in a first aspect to a polypeptide comprising an amino acid sequence selected from the group consisting of: SEQ ID NO:1, SEQ ID NO: 2 SEQ ID NO:3, SEQ ID NO:4, SEQ ID NO:5, SEQ ID NO:6, SEQ ID NO:7, and SEQ ID NO:8, or a variant sequence thereof, wherein said variant sequence exhibits at least 80% sequence identity, more preferably at least 85% sequence identity, most preferably at least 90% sequence identity with a sequence selected from the group consisting of: SEQ ID NO:1, SEQ ID NO: 2, SEQ ID NO:3, SEQ ID NO:4, SEQ ID NO:5, SEQ ID NO:6, SEQ ID NO:7 and SEQ ID NO:8. The polypeptide is preferably at most 643, preferably at most 625 amino acids long. SEQ ID NO:1 is a sequence motif conserved within mouse, rat and human GluN2A protein and is part of the C-terminal cytoplasmic domain thereof. Said 18 amino acid long sequence element is termed herein “canonical” GluN2A, because it is the “canonical” isoform found in the respective database entry of the UniProt database for the full length GluN2A protein (UniProtKB-Q12879; NMDE1_HUMAN; sequence version 1 of 1 Nov. 1996). The sequence corresponding to SEQ ID NO:1 in Grin2B protein is SEQ ID NO:7. Said 18 amino acid long sequence element is termed herein “canonical” GluN2B, because it is the “canonical” isoform found in the respective database entry of the UniProt database (UniProtKB-Q13224; NMDE2_HUMAN; sequence version 3 of 20 Jun. 2001). The inventors of the present invention have shown that polypeptides comprising these sequences are sufficient to induce NMDA receptor-mediated toxicity. SEQ ID NO: 2 and SEQ ID NO:3 are artificially created, mutated versions of SEQ ID NO:1 (i.e., derive from GluN2A), which still show some toxic properties. SEQ ID NO:4, SEQ ID NO:5 and SEQ ID NO:6 are naturally occurring variants of SEQ ID NO:1 (i.e., derive from GluN2A) occurring in the humans. Polypeptides comprising this sequence are also toxic and induce NMDA receptor mediated toxicity.

In preferred embodiments, the inventive polypeptide according to the present invention comprises aside of the above-mentioned core motif of the present invention also N-terminally and/or C-terminal flanking regions of the GluN2A- or GluN2B protein. For example, the polypeptide may comprise up to 1, 2, 3, 4, 5, 6, 7, 8, 9, 10 or 11 (or even more) additional amino acids of GluN2A or GluN2B, respectively, which are found directly adjacent N-terminal to the canonical sequence motif of SEQ ID NO:1 or SEQ ID NO:7. The polypeptide of the invention may for example also, or in addition, comprise up to 1, 2, 3, or 4 (or even more) additional amino acids of GluN2A, or GluN2B, which are found directly adjacent C-terminal to the inventive sequence motif of SEQ ID NO:1 or SEQ ID NO:7 in these proteins. Thus, a polypeptide according to the first aspect of the invention may comprise for example an amino acid sequence selected from the group consisting of SEQ ID NO:9, SEQ ID NO: 10 SEQ ID NO:11, SEQ ID NO:12, SEQ ID NO:13 and SEQ ID NO:14 in the context of GluN2A. In the context of GluN2B, the polypeptide of the first aspect of the invention may for example comprise a sequence according to SEQ ID NO:15 or a sequence according to SEQ ID NO:16. An illustration of these sequences and their structural relationship is provided in FIG. 1. Polypeptides according to the first aspect of the invention can be used—without being limited thereto—for studying and inducing NMDA receptor mediated toxicity in, e.g., neuronal cells, in particular regulation and interactions involved in said process.

The canonical amino acid sequence motif of GluN2A and GluN2B illustrated in SEQ ID NO:1 and SEQ ID NO:7 is not unique to humans but is highly conserved in mammals and even across vertebrates, as shown in tables 1 and 2 below:

TABLE 1
Sequence identity of inventive GluN2A
motif (SEQ ID NO: 1) in vertebrates

| | Species | Class | Identity |
|---|---|---|---|
| 1 | Callorhinchus milii | Chondrichthyes | 78% |
| 2 | Rhincodon typus | | 83% |
| 3 | Larimichthys crocea | Osteichthyes | 94% |
| 4 | Oncorhynchus mykiss | | 94% |
| 5 | Xiphophorus maculatus | | 100% |
| 6 | Nanorana parkeri | Amphibia | 100% |
| 7 | Xenopus laevis | | 100% |
| 8 | Xenopus tropicalis | | 100% |
| 9 | Anolis carolinensis | Reptilia | 100% |
| 10 | Pogona vitticeps | | 100% |
| 11 | Protobothrops mucrosquamatus | | 100% |
| 12 | Amazona aestiva | Aves | 100% |
| 13 | Columba livia | | 100% |
| 14 | Dromaius novaehollandiae | | 100% |
| 15 | Gallus gallus | | 100% |
| 16 | Parus major | | 100% |
| 17 | Pygoscelis adeliae | | 100% |
| 18 | Bos Taurus | Mammalia | 100% |
| 19 | Canis lupus familiaris | | 100% |
| 20 | Cavia porcellus | | 100% |
| 21 | Cricetulus griseus | | 100% |
| 22 | Equus caballus | | 100% |
| 23 | Erinaceus europaeus | | 100% |
| 24 | Felis catus | | 100% |
| 25 | Gorilla gorilla gorilla | | 100% |
| 26 | Heterocephalus glaber | | 100% |
| 27 | Homo sapiens | | 100% |
| 28 | Ictidomys tridecemlineatus | | 100% |
| 29 | Macaca nemestrina | | 100% |
| 30 | Mesocricetus auratus | | 100% |
| 31 | Mus caroli | | 100% |
| 32 | Mus musculus | | 100% |
| 33 | Ovis aries | | 100% |
| 34 | Pan troglodytes | | 100% |
| 35 | Rattus norvegicus | | 100% |
| 36 | Sus scrofa | | 100% |

TABLE 2
Sequence identity of inventive GluN2B
motif (SEQ ID NO: 7) in vertebrates

| | Species | Class | Identity |
|---|---|---|---|
| 1 | Callorhinchus milli | Chondrichthyes | 89% |
| 2 | Rhincodon typus | | 78% |
| 3 | Larimichthys crocea | Osteichthyes | 94% |
| 4 | Oncorhynchus mykiss | | 94% |
| 5 | Xiphophorus maculatus | | 94% |
| 6 | Nanorana parkeri | Amphibia | 89% |
| 7 | Xenopus laevis | | 94% |
| 8 | Xenopus tropicalis | | 89% |
| 9 | Anolis carolinensis | Reptilia | 94% |
| 10 | Pogona vitticeps | | 94% |
| 11 | Protobothrops mucrosquamatus | | 94% |
| 12 | Amazona aestiva | Aves | 100% |
| 13 | Columba livia | | 100% |
| 14 | Dromaius novaehollandiae | | 100% |
| 15 | Gallus gallus | | 100% |
| 16 | Picoides pubescens | | 100% |
| 17 | Pygoscelis adeliae | | 100% |
| 18 | Bos Taurus | Mammalia | 100% |
| 19 | Canis lupus familiaris | | 100% |
| 20 | Cavia porcellus | | 100% |
| 21 | Cricetulus griseus | | 100% |
| 22 | Equus asinus | | 100% |
| 23 | Erinaceus europaeus | | 100% |
| 24 | Felis catus | | 100% |
| 25 | Gorilla gorilla gorilla | | 100% |
| 26 | Heterocephalus glaber | | 100% |
| 27 | Homo sapiens | | 100% |
| 28 | Ictidomys tridecemlineatus | | 100% |
| 29 | Macaca mulatta | | 100% |
| 30 | Mesocricetus auratus | | 100% |
| 31 | Mus caroli | | 100% |
| 32 | Mus musculus | | 100% |
| 33 | Ovis aries | | 100% |
| 34 | Pan troglodytes | | 100% |
| 35 | Rattus norvegicus | | 100% |
| 36 | Sus scrofa | | 100% |

As evident from tables 1 and 2 above, the motif of interest is well conserved across various vertebrate and in particular mammalian species. It is therefore readily assumed that results obtained for one vertebrate, in particular mammalian species such as mouse or rat, are applicable to other vertebrate species, in particular mammalian species, as well. In this context, the inventors have shown that the sequence originally derived from mouse GluN2A/GluN2B can be used in both human HEK293 cells and in mouse primary cultured neurons, indicating the conserved function, and thus utility, of the inventive polypeptide across vertebrate and in particular mammalian species borders.

Preferably, the polypeptide of the present invention is toxic, preferably to both post-mitotic neurons and mitotic cancer cells where preferably TRPM4 protein is expressed. As used herein, a compound is “toxic”, if said compound induces both in vitro and in vivo cell death. A standard in vitro test involves treatment with or expression of these polypeptides in primary neurons for 10 days or in cell lines such as neuroblastoma cells, followed by assessments of cell death (see for example FIG. 2, and FIG. 5). A standard in vivo test is assessing neuronal death by staining degenerating neurons (e.g. with FluoJade C staining) after expressing inventive polypeptides for 3 weeks. Statistically relevant differences in the rate of cell death measured in vitro or in vivo compared to appropriate controls (i.e., saline solution, solvent only, inactive mutants) indicate toxicity.

The length of a polypeptide according to the first aspect of the present invention will preferably not exceed 625 amino acids in length. Although not limited thereto, this length would allow using essentially the entire cytoplasmic domain of GluN2A or GluN2B. However, in most cases the inventive polypeptide will be shorter. The polypeptide may for instance be at most about 625 amino acids long, at most about 600 amino acids long, at most about 500 amino acids long, at most about 400 amino acids long, at most about 300 amino acids long, at most about 250 amino acids long, at most about 200 amino acids long, at most about 150 amino acids long, at most about 125 amino acids long, at most about 100 amino acids long, at most about 90 amino acids long, at most about 85 amino acids long, at most about 80 amino acids long, at most about 75 amino acids long, at most about 70 amino acids long, at most about 65 amino acids long, at most about 60 amino acids long, at most about 55 amino acids long, at most about 50 amino acids long, at most about 45 amino acids long, at most about 40 amino acids long, at most about 35 amino acids long, at most about 30 amino acids long, at most about 25 amino acids long, at most about 20 amino acids long. In a preferred embodiment the inventive polypeptide is 18 to about 30 amino acids long. It is noted that the inventive polypeptide is not understood to be limited to fragments of GluN2A or GluN2B, although such constellation is the preferred embodiment of the polypeptide according to the first aspect of the invention.

In a second aspect, the present invention relates to a fusion protein comprising the inventive polypeptide according to the first aspect of the invention and at least one further amino acid sequence heterologous to the amino acid sequence according to the first aspect of the present invention. “Heterologous” implies, that the resulting fusion protein does not exist in this form in nature. This may be the case for example where the fusion protein represents a shuffled GluN2A or GluN2B variant in which GluN2A and/or GluN2B sequences, e.g. of different species, have been combined in a new and artificially created isoform (including mixed forms of GluN2A and GluN2B sequences), or, in a more preferred embodiment, because the further amino acid sequence is not at all related to a GluN2A or GluN2B protein. The term “fusion protein” does not imply a specific length of the polypeptide resulting from the fusion. Without being limited thereto, the at least one further amino acid sequence may be at least 5, at least 10, at least 15, at least 20, at least 25, at least 30, at least 50, at least 100 amino acids, at least 250 amino acids, or at least 500 or more amino acids in length. Typically, but not necessarily, the further amino acid sequence will provide an additional functionality to the polypeptide/resulting fusion protein. For example, the further amino acid sequence may be selected from the group consisting of a membrane anchoring moiety, a protein transduction domain and a tag. A membrane anchoring moiety is particularly useful if the fusion protein, and thus the polypeptide of the invention, is to be expressed in a cell, because GluN2A and GluN2B are membrane anchored receptors. Particularly preferred membrane anchoring moieties are selected from the group consisting of a CaaX box motif (for prenylation), a Glycosylphosphatidylinositol (GPI) signal anchor sequence (SEQ ID NO:17) and C-terminal targeting signal of K-Ras4B (Ras) protein (SEQ ID NO: 18). Regarding the CaaX box motif: C is the cysteine that is prenylated, a is any aliphatic amino acid, and the identity of X determines which enzyme shall act on the protein. Farnesyltransferase recognizes CaaX boxes where X=M, S, Q, A, or C, whereas Geranylgeranyltransferase I recognizes CaaX boxes with X=L or E. However, the inventive polypeptide may also be administered to cells for example from the outside by fusing it to a protein transduction domain, i.e., peptide sequences which facilitate transport of the polypeptide across the cell membrane and thus allow the polypeptide to through the cell membrane and to enter the cytosol of the cell. A preferred protein transduction domain is the TAT protein according to SEQ ID NO: 19. In addition, or alternative, it may be useful to label the polypeptide of the invention with a tag to allow for example detection or purification of the polypeptide/fusion protein. A preferred tag is the HA tag (SEQ ID NO: 20), which did not affect the activity of the inventive polypeptide at all, or a fluorescent protein tag, such as Green fluorescent Protein (GFP). A fusion protein according to the second aspect of the present invention is also preferably toxic.

Without being limited thereto, the fusion protein according to the second aspect of the invention is preferably at most about 600 amino acids long, at most about 500 amino acids long, at most about 400 amino acids long, at most about 300 amino acids long, at most about 250 amino acids long, at most about 200 amino acids long, at most about 150 amino acids long, at most about 125 amino acids long, at most about 100 amino acids long, at most about 90 amino acids long, at most about 85 amino acids long, at most about 80 amino acids long, at most about 75 amino acids long, at most about 70 amino acids long, at most about 65 amino acids long, at most about 60 amino acids long, at most about 55 amino acids long, at most about 50 amino acids long, at most about 45 amino acids long, at most about 40 amino acids long, at most about 35 amino acids long, at most about 30 amino acids long, at most about 25 amino acids long, at most about 20 amino acids long.

In a third aspect, the present invention relates to a nucleic acid encoding one or more inventive polypeptides of the present invention according to the first aspect and/or one or more fusion proteins according to the second aspect of the invention. The inventive nucleic acid may take all forms conceivable for a nucleic acid. In particular, the nucleic acids according to the present invention may be RNA, DNA or hybrids thereof. They may be single-stranded or double-stranded. They may have the size of small transcripts or of entire genomes, such as a viral genome. As used herein, a nucleic acid encoding one or more inventive polypeptides or fusion proteins of the present invention may be a nucleic acid reflecting the sense strand. Likewise, the antisense strand is also encompassed by the scope of the term “encoding”. The nucleic acid may encompass a heterologous (i.e., non GluN2A or GluN2B) promotor for expression of the inventive polypeptide, such as a viral promotor or bacterial promotor. It is understood that a nucleic acid according to the present invention cannot encode a full length GluN2A or GluN2B gene (the polypeptide according to the first aspect of the invention is shorter and the fusion protein comprises an amino acid sequence heterologous to GluN2A and GluN2B).

In a fourth aspect, the present invention relates to a vector comprising a nucleic acid according to the present invention. Such vector may for example be an expression vector allowing for expression of an inventive polypeptide or fusion protein. Such vector may for example be a viral expression vector. Said expression may be constitutive or inducible. The vector may also be a cloning vector comprising the sequence of a nucleic acid of the current invention for cloning purposes.

In a fifth aspect, the present invention relates to a polypeptide, fusion protein, nucleic acid, and/or vector according to the invention for use in a method of treating a disease in a subject, in particular for use in a method of treating cancer in a subject. Preferably, the cancer is a cancer of nerve tissue such as neuroblastoma. The method of treating cancer may be a method of treating cancer by inducing NMDA receptor mediated cell death. It is noted that the terminology “inducing NMDA receptor mediated cell death” is used in the context of this aspect of the invention to define the technical effect to be achieved, namely the induction of the respective cell death pathways, which are typically induced by (extrasynaptic) NMDA receptor signaling and lead to death of the respective cells. The method comprises administering to the subject in need of treatment an effective amount of the polypeptide or fusion protein of the invention or a nucleic acid or vector providing for expression of such polypeptide or fusion protein in the subject in need of treatment. The subject is typically a vertebrate, preferably a mammal, more preferably a human subject. The route of administration may be any route of administration considered by the skilled person suitable for the respective cancer. Preferably, the polypeptide, fusion protein, nucleic acid, or vector is administered intratumorally.

In a sixth aspect, the present invention relates to a (preferably isolated) cell comprising a polypeptide, fusion protein, nucleic acid, and/or vector according to the present invention.

In a seventh aspect, the present invention relates to a (preferably isolated) cell recombinantly expressing a GluN2A and/or GluN2B protein, wherein the GluN2A or GluN2B protein, respectively, comprises instead of the canonical sequence motif of SEQ ID NO:1 (GluN2A) and SEQ ID NO:7, respectively, a variant sequence thereof, or wherein said aforementioned sequence motif is deleted. The variant sequence will typically exhibit about the same sequence length as SEQ ID NO:1 (GluN2A) and SEQ ID NO:7, for example about 16 to about 20 amino acids, preferably about 17 to 19 amino acids, most preferably 18 amino acids. The GluN2A/GluN2B proteins of the cell of this aspect of the invention exhibit preferably as compared to canonical GluN2A and GluN2B, respectively, a reduced level of toxic activity. For instance, the variant sequence may exhibit one or more of the following variant amino acid residues in the sequence of SEQ ID NO:1: I3A, I7A, I11A, H15A. With respect to SEQ ID NO:7, the variant sequence may for example exhibit one or more of the following variant amino acid residues: I7A, I11A, H12R, A15H. Examples of GluN2A and GluN2B proteins, respectively, which can be used in the context of the seventh aspect of the invention, are GluN2A and/or GluN2B proteins exhibiting one of the following variant sequences: SEQ ID NO: 2, SEQ ID NO:3, SEQ ID NO:21, and SEQ ID NO:22. The variant sequence may of course be surrounded again by flanking sequences of GluN2A and GluN2B, respectively. GluN2A proteins of this aspect of the invention may therefore comprise for example one of the following sequences: SEQ ID NO:23 (i.e., GluN2A deletion mutant), SEQ ID NO: 10, SEQ ID NO:11, and SEQ ID NO:24. GluN2B proteins of this aspect of the invention may for example comprise one of the following sequences: SEQ ID NO:25 (i.e., GluN2B deletion mutant), SEQ ID NO:26, SEQ ID NO:27, and SEQ ID NO:28. “Recombinantly expressing a GluN2A and/or GluN2B protein”, as used herein, is intended to encompass all forms of artificially induced expression of GluN2A and/or GluN2B expression, be it now transiently or constitutively. For example, the GluN2A and/or GluN2B sequence may have been provided artificially to the cell by way of genetic engineering. The GluN2A or GluN2B protein may be derived from any species, but is preferably derived from a mammalian species, preferably mouse or human GluN2A or GluN2B, most preferably human GluN2A or GluN2B. It is understood that this pertains to the remainder of the GluN2A/GluN2B sequence, not the variant sequence of SEQ ID NO:1 (GluN2A) and SEQ ID NO:7, or the deletion mutants thereof. The term “GluN2A and/or GluN2B protein” also encompasses all variants of a GluN2A or GluN2B protein in a given species, i.e., is not limited to the canonical sequence of said species. It is noted that the GluN2A and/or GluN2B protein of this aspect of the invention preferably retains the capacity of forming a glutamate- and voltage-gated ion channel and to initiate physiological changes in the efficacy of synaptic transmission, triggering calcium signaling pathways to the cell nucleus and/or to activate neuronal structure-protective and survival-promoting genes.

The cell of the sixth and seventh aspect of the invention may be selected in particular from the group consisting of bacterial cells and yeast cells (e.g., for production purposes) as well as mammalian cells (e.g., for research purposes, but possibly also for production and other purposes). Mammalian cells may be neuronal but also non-neuronal mammalian cells. In a particularly preferred embodiment the cells are human embryonic kidney 293 (HEK293) cells. Such HEK293 cells may comprise for example GluN2A and/or GluN2B proteins as set out above.

In an eighth aspect, the present invention relates to a composition comprising a polypeptide, a fusion protein, a nucleic acid, a vector, or a cell (according to sixth or seventh aspect) of the invention. The composition may further comprise a pharmaceutically acceptable carrier, diluent or excipient. In a preferred embodiment, the composition may comprise a nanoparticle comprising said polypeptide, fusion protein, nucleic acid, vector and/or cell according to the present invention. The nanoparticle may be designed to release said polypeptide, fusion protein, nucleic acid, vector and/or cell over time.

In a ninth aspect, the present invention relates to an antibody, nanobody or anticalin, in particular an antibody or nanobody, binding to the C-terminal cytoplasmic domain of a GluN2A or GluN2B protein, respectively, but not binding to a GluN2A or GluN2B deletion mutant lacking the region corresponding to amino SEQ ID NO:1 and SEQ ID NO:7, respectively, (i.e., amino acids 861-878 of UniProtKB entry-Q12879; NMDE1_HUMAN; sequence version 1 of 1 Nov. 1996; and amino acids 862-879 of GluN2B; UniProtKB-Q13224; NMDE2_HUMAN; sequence version 3 of 20 Jun. 2001). Such antibody, nanobody or anticalin will be specific for the inventive region of GluN2A and GluN2B protein, respectively, which has been identified by the inventors to be relevant for induction of NMDA receptor mediated toxicity. The antibody may, for example, bind to a polypeptide sequence selected from the group consisting of: SEQ ID NO:1, SEQ ID NO: 2, SEQ ID NO:3, SEQ ID NO:4, SEQ ID NO:5, SEQ ID NO:6, SEQ ID NO:7, SEQ ID NO:8, SEQ ID NO:9, SEQ ID NO: 10, SEQ ID NO: 11, SEQ ID NO:12, SEQ ID NO:13, SEQ ID NO:14, SEQ ID NO:15, SEQ ID NO:16, SEQ ID NO:21, SEQ ID NO:22, SEQ ID NO:24, SEQ ID NO:26, and SEQ ID NO:27. Preferably, the antibody, nanobody or anticalin protects against, induces or ameliorates NMDA receptor mediated toxicity (for example, NMDA or glutamate induced neuronal death, oxygen glucose deprivation induced neuronal death, animal model of stroke), e.g., by preventing interaction of said subregion of GluN2A and GluN2B with other proteins or interaction partners involved in triggering NMDA receptor mediated toxicity, in particular TRPM4 protein. Such antibody, nanobody or anticalin could be considered an antagonist or blocking antibody, nanobody or anticalin, respectively. As used herein, a nanobody is a single-domain antibody (sdAb), i.e. an antibody fragment consisting of a single monomeric variable antibody domain.

In a tenth aspect, the present invention relates to the use of a polypeptide, a fusion protein, a nucleic acid, a vector, a cell, a composition and/or an antibody, nanobody or anticalin of the present invention for studying NMDA receptor mediated toxicity. As demonstrated in the examples of the present application, the inventors of the present invention identified the key region within GluN2A protein and GluN2B protein sufficient for induction of NMDA receptor mediated toxicity. This will allow scientists and researchers to study in more detail the molecular basis of events triggering NMDA receptor mediated toxicity.

In an eleventh aspect, the present invention relates to a method of sequencing in a genomic nucleic acid sample of a subject a partial sequence of the GRIN2A gene (i.e., the gene encoding GluN2A protein) and/or a partial sequence of the GRIN2B gene (i.e., the gene encoding GluN2B protein), wherein the sequenced partial sequence of GRIN2A gene comprises at least the gene region usually encoding the canonical sequence of SEQ ID NO:1 (i.e., amino acids 861-878 of human GluN2A) protein, and wherein the sequenced partial sequence of GRIN2B gene comprises the gene region usually encoding the canonical sequence of SEQ ID NO:7 (i.e., amino acids 862-879 of human GluN2B protein). The inventors of the present invention have shown that there are aside of the canonical sequence also variants (i.e., types) of GluN2A protein and GluN2B protein in the gene pool (e.g. of humans), and that depending on the genetic variant (i.e., type) of GluN2A and/or GluN2B, the susceptibility to NMDA receptor mediated toxicity varies. Therefore, determining the type of GluN2A and/or GluN2B can have clinical relevance and the method of this eleventh aspect of the invention is thus intended to provide sequence information about the GluN2A and GluN2B type of the subject (with respect to the inventive sequence in the cytoplasmic domain of GluN2A and GluN2B, respectively).

Preferably, the sequenced partial sequence of GRIN2A gene and/or GRIN2B gene covers at most 100, even more preferably at most 75, even more preferably at most 50 amino acids of the encoded GluN2A and GluN2B protein. Preferably, the sequencing makes use of polymerase chain reaction-based amplification and sequencing. It is noted that in the human genome, the inventive amino acids sequence motif is distributed over two exons, i.e., is interrupted by an intron. Therefore, the means to sequence said region will need to consider this fact. The method of the eleventh aspect of the invention may, for example, be carried out by sequencing the two appropriate regions at the exon-intron boundaries. In the case of PCR using standard primers, suitable primers for amplification of the GRIN2A target sequence in the human genome are for example provided in SEQ ID NO:29 and SEQ ID NO:30 (forward and reverse primer of GRIN2A, 1^{st} region) and SEQ ID NO:31 and SEQ ID NO:32 (forward and reverse primer of GRIN2A, 2^{nd} region). Said amplified nucleic acids can then be sequenced with the sequencing primers represented by SEQ ID NO:33 and SEQ ID NO:34). For the GRIN2B target sequence in the human genome, SEQ ID NO:35 and SEQ ID NO:36 are suitable forward and reverse primers for the 1^{st} region and SEQ ID NO:37 and SEQ ID NO:38 are suitable forward and reverse primers for the 2^{nd} region. Said amplified nucleic acid can then for example be sequenced with the sequencing primers represented by SEQ ID NO:39 and SEQ ID NO:40. The method of the eleventh aspect of the invention is preferably suited to allow—on basis of the sequenced genomic nucleic acid sample—determining whether or not the subject exhibits one or more of the following (all position indications provided relative to the sequence of UniProtKB-Q12879 or UniProtKB-Q13224):
canonical GluN2A (see also SEQ ID NO:1),
GluN2A (I867M) (see also SEQ ID NO:4),
GluN2A (S869R) (see also SEQ ID NO:5),
GluN2A (I876N) (see also SEQ ID NO:6),
canonical GluN2B (see also SEQ ID NO:7),
GluN2B (I872M) (see also SEQ ID NO:8),
GluN2B (H873R) (see also SEQ ID NO:22).

The inventors of the present invention found out that the above-mentioned GluN2A and GluN2B variants (caused by different SNPs in the respective genes) lead to a different level of susceptibility to NMDA receptor mediated toxicity in HEK293 cell lines, as demonstrated in the examples. The above-mentioned method of the eleventh aspect of the invention may thus also be a method for assessing the susceptibility of a subject to NMDA receptor mediated toxicity, the method comprising sequencing in a genomic nucleic acid sample of a subject a partial sequence of the GRIN2A gene (i.e., the gene encoding GluN2A protein) and/or a partial sequence of the GRIN2B gene (i.e., the gene encoding GluN2B protein), wherein the sequenced partial sequence of GRIN2A gene comprises at least the gene region usually encoding the canonical sequence of SEQ ID NO:1 (i.e., amino acids 861-878 of human GluN2A) protein, and wherein the sequenced partial sequence of GRIN2B gene comprises the gene region usually encoding the canonical sequence of SEQ ID NO:7 (i.e., amino acids 862-879 of human GluN2B protein), and wherein a subject with:
a) a canonical GluN2A (SEQ ID NO:1) and/or a canonical GluN2B (SEQ ID NO:7) sequence exhibit a regular susceptibility to NMDA receptor mediated toxicity,
b) a GluN2A (I867M) mutation (see also SEQ ID NO:4) exhibits an increased susceptibility to NMDA receptor mediated toxicity,
c) a GluN2A (S869R) mutation (see also SEQ ID NO:5) exhibits a regular susceptibility to NMDA receptor mediated toxicity,
d) a GluN2A (I876N) mutation (see also SEQ ID NO:6) exhibits an increased susceptibility to NMDA receptor mediated toxicity,
e) a GluN2B (I872M) mutation (see also SEQ ID NO:8) exhibits a regular susceptibility to NMDA receptor mediated toxicity, and
f) a GluN2B (H873R) mutation (see also SEQ ID NO:22) exhibits a decreased susceptibility to NMDA receptor mediated toxicity.

Preferably, the method of the eleventh aspect of the invention is an ex vivo method, i.e., is not carried out on the human or animal body. The subject providing the sample is preferably a mammal, preferably selected from the group consisting of human, mouse, rat, dog, cat, cow, monkey, horse, hamster, guinea pig, pig, sheep, goat, rabbit etc. Most preferably, the subject is a human being.

In a further, twelfth aspect, the present invention relates to the use of a polypeptide, a fusion protein according to the present invention or a cell according to the present invention in protein-protein interaction assays. While the inventors disclose herein the importance of the inventive region in the GluN2A and GluN2B protein for NMDA receptor mediated toxicity, it is not clear which other protein factors may be involved in promoting or restricting NMDA receptor mediated toxicity. It is contemplated that a polypeptide/fusion protein according the first and second aspect of the invention (or a cell according to the sixth or seventh aspect) will be particularly useful for identifying further binding and interaction partners of GluN2A or GluN2B proteins. The protein-protein interaction under scrutiny in such assays is preferably an interaction within the region specified by the amino acid sequence according to SEQ ID NO:1 and/or SEQ ID NO:7, or their variant sequences as defined herein. Such binding partners may turn out to be neurotoxic, neuroprotective or neither thereof. In this context the polypeptide/fusion protein/cell will not only provide insights regarding direct interaction partners but will also shed light on interactions of other compounds involved in GluN2A/GluN2B signaling, for example if certain complexes can no longer be formed due to (e.g., competitive) inhibition. The person skilled in the art is familiar with a large number of possible assays for determining protein-protein interactions, including biochemical, biophysical and genetic methods. Non-limiting examples are immunoprecipitation, bimolecular fluorescence complementation (e.g. split-TEV, split-GFP), affinity electrophoreses, immunoelectrophoresis, phage display, tandem affinity purification, chemical crosslinking followed by mass spectrometry analysis, surface plasmon resonance, fluorescence resonance energy transfer, nuclear magnetic resonance imaging, reverse assays where binding to the SEQ ID NO:1 and/or SEQ ID NO:7 abolishes the signals in above mentioned assays, complementation assays where a compound library is added to non-responder cells to make them responsive or RNAi screening assays (to make responder cells on-responsive) etc. The protein-protein interaction assay may be an in vitro, ex vivo or an in vivo assay. Most preferably, the protein-protein interaction assay is an in vitro assay. However, in the context of live imaging such assay may also be an in vivo assay. In cases where the assay is an in vivo assay, it is preferably not an assay in a human being.

Potential interactions with the inventive region may not only be determined by conventional wet chemistry means but also by novel in silico approaches. Therefore, the present invention also relates in a thirteenth aspect to a method for identifying a compound potentially interacting with the C-terminal cytoplasmic domain of a GluN2A or GluN2B protein, wherein the method comprises:
i) computer-assisted virtual docking of a candidate compound to an amino acid sequence according to SEQ ID NO:1 or SEQ ID NO:7 or to a variant thereof, the variant being selected from GluN2A (I867M) (see also SEQ ID NO:4), GluN2A (S869R) (see also SEQ ID NO:5), GluN2A (I876N) (see also SEQ ID NO:6), GluN2B (I872M) (see also SEQ ID NO:8), and GluN2B (H873R) (see also SEQ ID NO:22), wherein said amino acid sequence is provided in a virtual 3-D structure of a polypeptide comprising said amino acid sequence, and
ii) determining the docking score and/or internal strain for docking the candidate compound virtually to the amino acid sequence according to SEQ ID NO:1 or SEQ ID NO:7 or its variant, and optionally
iii) contacting in vitro or in vivo an N-methyl-D-aspartate (NMDA) receptor comprising a GluN2A or GluN2B subunit, respectively, with the candidate compound to determine whether the candidate compound modulates the activity of said NMDA receptor or not.

As already mentioned above, methods to determine NMDA receptor activity are known in the art (see for example Zhang et al., 2011, J. Neurosci. 31, 4978-4990; incorporated herewith by reference).

Methods for in silico docking of candidate compounds to protein structures are well known in the art. The candidate compound can be any compound. Typically, the compound will be a small molecule, but it is also possible that the compound is a large biomolecule, such as a protein (e.g., an antibody or the like). Collections of compounds are for example available from Schrödinger LLC (New York, NY, USA). The 3D structure can be any structure comprising an amino acid sequence according to SEQ ID NO:1 or SEQ ID NO:7 or variant thereof (SEQ ID NO:2, SEQ ID NO:3, SEQ ID NO:4, SEQ ID NO:5, SEQ ID NO:6, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:21, SEQ ID NO:22, SEQ ID NO:24, SEQ ID NO:26, SEQ ID NO:27 and SEQ ID NO:28). The 3D structure can be any 3D structure of any vertebrate GluN2A or GluN2B protein or of parts of any of these comprising the inventive sequence motif. Preferably, the 3D structure is of mammalian origin, even more preferably of human origin. The 3D structure may also be a structure of a complex comprising the GluN2A or GluN2B protein, for example a complex of GluN1 and GluN2A or GluN2B. In the present case, various structures of GluN2A and GluN2B proteins are available to the skilled person, for example in the Protein Data Bank, and can be used for the method of the thirteenth aspect of the invention. The 3D structure may be based for example on a structured obtained by x-ray crystallography analysis, NMR spectroscopy analysis, cryo-EM, or derived from homology modeling. It is understood that the method according to the thirteenth aspect of the invention will encompass docking of a candidate compound to the region in the GluN2A/GluN2B structure, which corresponds to the amino acid sequence according to SEQ ID NO:1 or SEQ ID NO:7 or variant thereof (in particular those described above), and does not encompass docking to regions of the structure, which do not relate to these amino acid sequences. However, if docking to the region with SEQ ID NO:1 or SEQ ID NO:7 or variant thereof requires in parallel interaction with other amino acid residues outside said region, then such docking is also encompassed by the method according to the thirteenth aspect of the invention. Docking itself can be accomplished by a variety of methods known to the person skilled in the art and respective software is publicly available (see for example Schrödinger LLC, New York, NY, USA). A respective analysis can also be ordered from commercial providers, for example Proteros biostructures GmbH (Planegg, Germany). The method of the thirteenth aspect of the present invention may also be used to identify inhibitors of NMDA receptor mediated toxicity.

In a further, fourteenth aspect, the present invention relates to a method of providing a cell with a nucleic acid, in particular a gene, providing more resistance towards NMDA receptor mediated toxicity, the method comprising replacing in said cell a GRIN2A and/or GRIN2B genomic sequence linked with a higher susceptibility towards NMDA receptor mediated toxicity with a GRIN2A and/or GRIN2B sequence linked with a lower susceptibility towards NMDA receptor mediated toxicity. For example, a GRIN2A genomic sequence linked with a (e.g., compared to canonical GRIN2A/GRIN2B) high susceptibility (see for instance a GRIN2A gene variant encoding the I876N subtype; see also SEQ ID NO:6) towards NMDA receptor mediated toxicity may be replaced with a GRIN2A canonical genomic sequence or with a sequence providing for an even lower susceptibility towards NMDA receptor mediated toxicity than the canonical sequence (for instance a GRIN2A gene variant encoding a deletion mutant; see also SEQ ID NO:23; or the sequences according to SEQ ID NO:2, SEQ ID NO:3 or SEQ ID NO:21). Similarly, a canonical GRIN2B genomic sequence may be replaced with a sequence providing for a lower susceptibility towards NMDA receptor mediated toxicity than the canonical sequence (for instance a GRIN2B gene variant encoding a deletion mutant; see also SEQ ID NO:25; or the sequences according to SEQ ID NO:2, SEQ ID NO:3, or SEQ ID NO:22). The method is preferably an in vitro method. The cell may be for example a neuronal cell, a stem cell, in particular an embryonic stem cell, an oocyte or a spermatocyte. The cell is preferably of mammalian origin, even more preferably of human origin. However, non-human mammalian cells are also specifically contemplated by the invention. In preferred embodiments, the nucleic acid (or gene) providing more resistance towards NMDA receptor mediated toxicity may be a nucleic acid (or gene) entirely lacking the inventive sequence motif region (i.e., deletion mutants). In a preferred embodiment the gene replacement is achieved by CRISPR or other techniques mediated gene editing. The replacement will render neurons more resistant against NMDA toxicity and consequently against many forms of neurodegenerations (including stroke, ALS, Alzheimer Disease, Huntington Disease, retina degenerations (glaucoma, diabetic retinopathy), etc.).

In a fifteenth aspect, and in analogy to the above mentioned fourteenth aspect, the present inventions relate to a method of site-directed messenger RNA (mRNA) editing of cells, organs, rodents, mammals, or humans within the inventive region of both GluN2A and GluN2B, providing the host more resistance towards NMDA receptor mediated toxicity by altering an mRNA linked with a higher susceptibility towards NMDA receptor mediated toxicity into a GRIN2A and/or GRIN2B mRNA sequence linked with a lower susceptibility towards NMDA receptor mediated toxicity. Site-directed mRNA editing is accomplished with the enzyme adenosine deaminase acting on RNA (ADAR) that catalyzes the deamination of the nucleotide adenosine to inosine. Inosine is structurally similar to guanine, mimics guanosine during translation, and can result in changes in the protein coding sequence. Several techniques for site-directed mRNA editing are available (Aquino-Jarquin, 2020, Mol Ther Nucleic Acids 19, 1065-1072; incorporated herein by reference). For example, a deaminase domain of a human ADAR fused to SNAP tag and a short (about 20-nt) guide RNA is being introduced into the cells by transfection or infection methods. The guide RNA binds to the ADAR-SNAP tag fusion protein and targets it to the desired mRNA. The guide RNA is designed to contain an A:C mismatch at the target site to induce site-specific deamination (Vogel et al., 2018, Nat Meth 15, 535-538; incorporated herein by reference). A simplified method requires transfection into the cells of chemically modified, stabilized antisense oligonucleotide (ASO) designed to contain two segments: a guide sequence that determines target mRNA binding and a ADAR recruiting domain to guide endogenous human ADARs to the ASO:mRNA hybrid to edit the transcripts (Merkle et al., 2019, Nat Biotech 37, 133-138; incorporated herein by reference). The desired changes in the inventive target sequence include but are not limited to the following amino acids (after editing: I becomes V, H becomes R): 1863V, 1867V, 1871V, 1876V, H875R and combinations thereof (all in GluN2A); I864V, I868V, I872V, I877V, H873R and combinations thereof (all in GluN2B). Preferably, the method of this aspect of the invention comprises editing or replacing in said cell a GluN2A and or GluN2B mRNA sequence linked with a higher susceptibility towards NMDA receptor mediated toxicity with a GluN2A or GluN2B mRNA sequence linked with a lower susceptibility towards NMDA receptor mediated toxicity (see also above). The method is an in vitro or in vivo method. The cell may be for example a neuronal cell, a stem cell, in particular an embryonic stem cell, an oocyte or a spermatocyte. The cell or organ is preferably of rodent or mammalian origin, even more preferably of human origin. However, non-human mammalian cells and organs are also specifically contemplated by the invention. The replacement or editing of the GluN2A or GluN2B mRNA sequence follows the pattern as set out above. For example, if the cell contains a mRNA encoding a GluN2B protein with the sequence motif according to SEQ ID NO:7, it may be replaced by a mRNA encoding a GluN2B protein with the mRNA motif according to SEQ ID NO:22. In a further example: if the cell comprises a sequence encoding a GluN2A protein with the sequence motif according to SEQ ID NO:6, it may be replaced for example by a sequence encoding a GluN2A protein with the sequence motif according to SEQ ID NO:1 or SEQ ID NO:2, SEQ ID NO:3, or SEQ ID NO:21, which all provide for a lower susceptibility to NMDA receptor mediated toxicity than SEQ ID NO:6. In some embodiments the messenger RNA (or gene) providing more resistance towards NMDA receptor mediated toxicity may be a messenger RNA (or gene) entirely lacking the inventive sequence motif (i.e., deletion mutants with the sequence corresponding to SEQ ID NO:1 or SEQ ID NO:7 being deleted). In a preferred embodiment the mRNA replacement is achieved by CRISPR or other methods mediated mRNA editing. As before, such mRNA replacement will render neurons more resistant against NMDA toxicity and consequently against many forms of neurodegenerations (including stroke, ALS, Alzheimer Disease, Huntington Disease, retina degenerations (glaucoma, diabetic retinopathy), etc.).

In a final, sixteenth aspect, the present invention relates to a kit comprising the means to determine in a genomic nucleic acid sample of a subject the sequence of a partial sequence of the GRIN2A gene (i.e., the gene encoding GluN2A protein) and/or a partial sequence of the GRIN2B gene (i.e., the gene encoding GluN2B protein), wherein the sequenced partial sequence of GRIN2A gene comprises at least the gene region usually encoding the canonical sequence of SEQ ID NO:1 (i.e., amino acids 861-878 of human GluN2A) protein, and wherein the sequenced partial sequence of GRIN2B gene comprises the gene region usually encoding the canonical sequence of SEQ ID NO:7 (i.e., amino acids 862-879 of human GluN2B protein). The means may for example be nucleic acid primers or probes allowing amplification, sequencing and/or detection of the respective partial sequence of GRIN2A gene and/or GRIN2B gene. Preferably, the means allow determining the partial sequence of GRIN2A gene and/or GRIN2B gene, the partial sequence coving at most 100, even more preferably at most 75, even more preferably at most 50 amino acids of the encoded GluN2A and GluN2B protein. Suitable means may for example be selected from the group of primers mentioned above, i.e., SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, and SEQ ID NO:40. The kit may be a kit for performing the method according to the eleventh aspect of the invention. The kit may allow—on basis of the sequenced genomic nucleic acid sample—to determine whether or not the subject exhibits one or more of the following (all position indications provided relative to the sequence of UniProtKB-Q12879 or UniProtKB-Q13224):
canonical GluN2A (see also SEQ ID NO:1),
GluN2A (I867M) (see also SEQ ID NO:4),
GluN2A (S869R) (see also SEQ ID NO:5),
GluN2A (I876N) (see also SEQ ID NO:6),
canonical GluN2B (see also SEQ ID NO:7),
GluN2B (I872M) (see also SEQ ID NO:8),
GluN2B (H873R) (see also SEQ ID NO:22).

Preferably, the kit comprises at least the means to identify the inventive sequence motif encoding canonical GluN2A (SEQ ID NO:1) and canonical GluN2B (SEQ ID NO:7). In another embodiment the kit may comprise at least the means to detect the inventive sequence motif encoding canonical GluN2A (SEQ ID NO:1) and one further variant of the inventive sequence motif of canonical GluN2A, selected for example from the group consisting of GluN2A (I867M), GluN2A (S869R), and GluN2A (I876N). In a further embodiment, the kit may comprise at least the means to detect the inventive sequence motif encoding canonical GluN2B (SEQ ID NO:7) and one further variant of the inventive sequence motif encoding canonical GluN2B, selected for example from the group consisting of GluN2B (1872M) and GluN2B (H873R). The subject providing the sample is preferably a mammal, preferably selected from the group consisting of human, mouse, rat, dog, cat, cow, monkey, horse, hamster, guinea pig, pig, sheep, goat, rabbit etc. Most preferably, the subject is a human being.

The term “comprising”, as used herein, shall not be construed as being limited to the meaning “consisting of” (i.e., excluding the presence of additional other matter). Rather, “comprising” implies that optionally additional matter may be present. The term “comprising” encompasses as particularly envisioned embodiments falling within its scope “consisting of” (i.e., excluding the presence of additional other matter) and “comprising but not consisting of” (i.e., requiring the presence of additional other matter), with the former being more preferred.

### FIGURES

In the following a brief description of the appended figures will be given. The figures are intended to illustrate aspects of the present invention in more detail.

FIG. 1 illustrates an alignment of the inventive sequence motif and variants thereof identified by the inventors. The inventive sequence core motif is situated in the cytoplasmic domain of GluN2A and GluN2B protein and has a length of 18 amino acids, containing four regularly-spaced isoleucine residues. The depicted flanking sequences are human sequences, which may be found, however, identically in rat and mouse GluN2A/GluN2B.
“-”: deleted amino acid residue; bold letters: deviation from canonical core sequence.
It is noted that the inventive core sequence motif is identical except for a histidine residue in GluN2A at position 15 of the core, whereas GluN2B exhibits at this position an alanine residue. Therefore, mutation of said GluN2B alanine residue into a histidine residue will yield the GluN2A sequence, but in the GluN2B framework, and vice versa.
A) GluN2A protein and variants thereof; wherein SEQ ID NO:1 represents the canonical inventive sequence motif in the GluN2A protein;
B) GluN2B protein and variants thereof: wherein SEQ ID NO:7 represents the canonical inventive sequence motif in the GluN2B protein.

FIG. 2 illustrates the impact of deletion or point mutation of the inventive sequence motif (in either GluN2A or GluN2B, respectively) on cell death of HEK293 cells. A) Controls: Neither untransfected cells nor cells transfected with GFP or GluN1 alone show induction of cell death. B) GluN2A: GluN1+GluN2A (wildtype receptor complex); GluN1+GluN2AΔSEQ ID NO:1 (deletion mutant; SEQ ID NO:41); GluN1+GluN2AΔ9 (control lacking last 9 C-terminal amino acids of GluN2A, which are not related to the inventive sequence motif); GluN1+GluN2A+MK-801 (wildtype receptor complex in the presence of the non-competitive, general NMDA receptor inhibitor, MK-801 C) GluN2B: GluN1+GluN2B (wildtype receptor complex); GluN1+GluN2BΔSEQ ID NO:7 (deletion mutant; SEQ ID NO:42); GluN1+GluN2BΔ9 (control lacking last 9 C-terminal amino acids of GluN2B, which are not related to the inventive sequence motif); GluN1+GluN2B+MK-801 (wildtype receptor complex in presence of the non-competitive, general NMDA receptor inhibitor, MK-801; n.s.: not significant; *: p<0.05; **: p<0.01.

FIG. 3 illustrates the capacity of GluN2A and GluN2B mutants to induce—in presence of GluN1—cell death in HEK293 cells 48 hours after transfection: from left to right: rat wild type GluN2A (UniProt: Q00959); GluN2AΔSEQ ID NO:1 (SEQ ID NO:41; i.e., deletion mutant lacking SEQ ID NO:1); GluN2A-I2A2 (like wild type GluN2A (UniProt: Q00959), but exhibiting the substitutions I867A and I871A (see also SEQ ID NO: 2); GluN2A-13A3 exhibiting the substitutions I863A, I867A and I871A (see SEQ ID NO:3); GluN2A-IAHA exhibiting the substitutions I867A, I871A and H875A (see SEQ ID NO:21); rat wild type GluN2B (UniProt: Q00960); GluN2BΔSEQ ID NO:7 lacking the inventive 18 amino acid sequence motif in GluN2B (see SEQ ID NO:42; i.e., a deletion mutant), GluN2B-12A2 (like wild type GluN2B (UniProt: Q00960), but exhibiting the substitutions I868A and I872A (see also SEQ ID NO:21); and GluN2B-I2A2AH (like wild type GluN2B (UniProt: Q00960), but exhibiting the substitutions I868A, I872A and A876H); *: p<0.05; **: p<0.01.

FIG. 4 illustrates the capacity of GluN2A and GluN2B variant sequences (occurring in the human gene pool) to induce in presence of GluN1 cell death in HEK293 cells 48 hours after transfection: from left to right: rat wild type GluN2A (UniProt: Q00959); GluN2A-1867M (like wild type GluN2A (UniProt: Q00959), but exhibiting the substitution I867M; see also SEQ ID NO:4); GluN2A-S869R (like wild type GluN2A (UniProt: Q00959), but exhibiting the substitution S869R; see also SEQ ID NO:5); GluN2A-1876N (like wild type GluN2A (UniProt: Q00959), but exhibiting the substitution I876N; see also SEQ ID NO:6); rat wild type GluN2B (UniProt: Q00960); GluN2B-1872M (like wild type GluN2B (UniProt: Q00960), but exhibiting the substitution I872N; see also SEQ ID NO:8); GluN2B-H873R (like wild type GluN2B (UniProt: Q00960), but exhibiting the substitution H873R; see also SEQ ID NO:8). *: p<0.05.

FIG. 5 illustrates that a GluN2A derived polypeptide (SEQ ID NO:9), comprising the sequence of SEQ ID NO:1, or a GluN2B derived polypeptide (SEQ ID NO:15) comprising the sequence of SEQ ID NO:7, is sufficient to induce cell death in mouse hippocampal and cortical neurons, while mutant versions (SEQ ID NO:10; comprising the sequence of SEQ ID NO:2; and SEQ ID NO:26; comprising the sequence of SEQ ID NO:21) are not able to induce cell death. ****: p<0.0001.

FIG. 6 illustrates a possible sequencing strategy for human GRIN2A and GRIN2B gene (i.e., the genes encoding GluN2A and GluN2B protein, respectively) on basis of the Genome Reference Consortium Human Build 38 patch release 13 (GRCh38.p13) for Homo sapiens chromosome 16. Long arrows refer to PCR primers, short arrows to sequencing primers. Black lines represent the inventive sequence region according to SEQ ID NO:1 and SEQ ID NO:7. As can be seen, said region are interrupted by an intron. A) GRIN2A: GRIN2A-1 region: 9768629 to 9769128, includes the first half of SEQ ID NO:1 (TGVCSDRPGLLFSISR; SEQ ID NO:43), the PCR (with primers GRIN2A-1-F, SEQ ID NO:29, and GRIN2A-1-R, SEQ ID NO:30) yields a 405 bp fragment. GRIN2A-2 region: 9764689 to 9765188, includes the second half of SEQ ID NO:1 (GIYSCIHGVHIEEKKKS; SEQ ID NO:44), the PCR (with primers GRIN2A-2-F, SEQ ID NO:31, and GRIN2A-2-R, SEQ ID NO:32) yields a 368 bp fragment B) GRIN2B: GRIN2B-1 region: 13566792 to 13567291, includes the first half of SEQ ID NO:7 (MGVCSGKPGMVFSISR; SEQ ID NO:45), the PCR with primers GRIN2B-1-F, SEQ ID NO:35, and GRIN2B-1-R, SEQ ID NO:36) yields a 455 bp fragment; GRIN2B-2 region: 13564382 to 13564881, includes the second half of SEQ ID NO:7 (GIYSCIHGVAIEERQSV; SEQ ID NO:46), the PCR with primers GRIN2B-2-F, SEQ ID NO:37, and GRIN2B-2-R, SEQ ID NO:38) will yield a 395 bp fragment.

### EXAMPLES

In the following specific examples illustrating embodiments and aspects of the invention are presented. However, the present invention shall not to be limited in scope by the specific examples described herein. Indeed, various modifications of the invention in addition to those described herein will become readily apparent to those skilled in the art from the foregoing description and the example below. All such modifications fall within the scope of the appended claims.

### Example 1: Methods and Materials

The following methods and materials were used by the inventors in the subsequent examples, unless indicated otherwise.

The original plasmids construct of GRIN1, GRIN2A and GRIN2B, which are genes encoding GluN2A and GluN2B, were gifts from Andres Barria & Robert Malinow (Addgene plasmid #45446, 45445, 45447). All the mutants in the application are generated using site directed mutagenesis by PCR. The primers used to generate the plasmids were as following:

### For GrinN2A:

| Gene | | Forward Primer | | |
|---|---|---|---|---|
| name | Template | (5′-3′) | Reverse Primer (5′-3′) | SEQ ID NO |
| GRIN2AΔ | GRIN2A | ccgggctgctccacatt | tcttcaatgtggagcagcccggg | 47, 48 |
| SEQ | | gaagaaaagaaga | ccggtca | |
| ID | | | | |
| NO: 1 | | | | |
| GRIN2AΔC9 | GRIN2A | cgctaaagagtcctagg | gtctgctcgaagcggccgcttact | 49, 50 |
| | | tatct | tgtacactcgtctattg | |
| GRIN2A- | GRIN2A | ctccatcagcaggggc | ccatggGCgcaactatagGCg | 51, 52 |
| I2A2 | | GCctatagttgcGCc | cccctgctgatggag | |
| | | catgg | | |
| GRIN2A- | GRIN2A- | ggctgctcttctccgcca | atagGCgcccctgctggcggag | 53, 54 |
| I3A3 | I2-A2 | gcaggggcGCctat | aagagcagcc | |
| GRIN2A- | GRIN2A- | tgcGCccatggggta | cttttcttcaatggctaccccatgg | 55, 56 |
| I2A2HA | I2-A2 | gccattgaagaaaag | GCgca | |
| GRIN2A- | GRIN2A | catcagcaggggcatgt | atggatgcaactatacatgcccct | 57, 58 |
| I867M | | atagttgcatccat | gctgatg | |
| GRIN2A- | GRIN2A | ggcatctatagatgcatc | ccatggatgcatctatagatgcc | 59, 60 |
| S869R | | catgg | | |
| GRIN2A- | GRIN2A | catccatggggtacaca | ttcttcaTtgtgtaccccatggatg | 61, 62 |
| I876N | | Atgaagaa | | |

### For GrinN2B:

| Gene | | Forward Primer | | |
|---|---|---|---|---|
| name | Template | (5′-3′) | Reverse Primer (5′-3′) | SEQ ID NO |
| GRIN2BΔ | GRIN2B | tggcatggtcgccatag | cctctatggcgaccatgccaggct | 63, 64 |
| SEQ | | aggagcgcca | tgcca | |
| ID | | | | |
| NO: 7 | | | | |
| GRIN2BΔC9 | GRIN2B | gtggattgggaggacc | ggcaagaattctcactcataaacat | 65, 66 |
| | | ggtctgg | gtccattg | |
| GRIN2B- | GRIN2B | ctccatcagcagaggtg | ccatgggcacagctgtaggcacc | 67, 68 |
| I2A2 | | cctacagctgtgcccat | tctgctgatggag | |
| | | gg | | |
| GRIN2B- | GRIN2B- | tgtgcccatggggtaC | ggogctcctctatgTGtacccca | 69, 70 |
| I2A2AH | I2-A2 | Acatagaggagcgcc | tgggcaca | |
| GRIN2B- | GRIN2B | atctacagctgtatgcat | ggctaccccatgcatacagctgta | 71, 72 |
| I872M | | ggggtagcc | gat | |
| GRIN2B- | GRIN2B | ctacagctgtatccgtg | tctatggctaccccacggatacag | 73, 74 |
| H873R | | gggtagccataga | ctgtag | |

HEK293 Cell Culture HEK293 cells were cultured in Dulbecco's Modified Eagle Medium (DMEM, Gibco™, 41965-039) supplement with 10% Fetal Bovine Serum (FBS, Gibco™, 10270), 1% Sodium Pyruvate (Gibco™, 11360070), 1% MEM NEAA (Gibco™, 11140035) and 0.5% Penicillin-Streptomycin (P-S; Sigma, P0781) and Passage15-25 were used for experiments.

### Luminescent Cytotoxicity Assay

To test the cytotoxicity of compounds according to the present invention, HEK293 cells (70-80% confluent) were transfected 24 hours after plating with expression vectors for both GluN1 and GluN2A or GluN2B, respectively (1:1; 2 μg plasmids in total per 6-cm dishes) with Lipofectamine 2000 according to manufacturer's instructions. The relative number of dead cells in the population at the indicated time points after transfection was measured with the CytoTox-Glo™ Cytotoxicity assay (Promega, G9290) according to manufacturer's instruction with minor modification. Briefly, 10% of total medium were mixed with 10 μL AAF-amino luciferin to reach a final volume at 200 μL with water, the dead cell relative luminescence units (DRLU) was measured by GloMax (Promega) in a 96 well white bottom polystyrene microplate (Corning Costar®, 3912). After all the measurements, lysis reagents have been added to the cells and 10% of lysate was used for the total cell relative luminescence units (TRLU) measurement. Cell death was calculated by the following equations:

$$Cell death ( \% ) = \frac{10 * D R L U}{10 * T R L U + D R L U} * 100$$

### Primary Neuronal Cultures

Primary mouse hippocampal were prepared and maintained as known to the skilled person. Briefly, hippocampi from P0 C57Bl/6N mice were dissociated and plated at a density of 1.2*105/cm² in Growth Medium (GM), consisting of Neurobasal A medium (Gibco™, 10888022), 2% serum free B27™ Supplement (Gibco™, 17504044), 1% rat serum (Biowest, S2150), 0.5 mM L-Glutamine (Sigma, G7513) and 0.5% P-S. Cytosine P-D-arabinofuranoside (AraC; Sigma, C1768; 2.8 μM) was added on DIV3 to prevent the proliferation of glial cells. From DIV8, half of the medium was replaced by GM without Rat serum every 48 h until being used for experiments. 24 h before experiments, GM was replaced with transfection medium (10 mM HEPES, pH 7.4, 114 mM NaCl, 26.1 mM NaHCO₃, 5.3 mM KCl, 1 mM MgCl₂, 2 mM CaCl₂), 30 mM glucose, 1 mM glycine, 0.5 mM C₃H₃NaO₃, and 0.001% phenol red and 10% of phosphate-free Eagle's minimum essential medium, supplemented with 7.5 μg/ml insulin, 7.5 μg/ml transferrin and 7.5 ng/ml sodium selenite (ITS Liquid Media Supplement, Sigma-Aldrich Cat #I3146)).

Recombinant Adeno-Associated Viruses (rAAVs) and Constructs

Viral particles were produced and purified as known in the art. The nucleic acid constructs expressing SEQ ID NO:9 (i.e., comprising SEQ ID NO:1), SEQ ID NO: 10 (i.e., comprising SEQ ID NO: 2), SEQ ID NO:15 (i.e., comprising SEQ ID NO:7), and SEQ ID NO:26 (i.e., comprising SEQ ID NO:3), were driven by a human synapsin promoter, fused to an HA tag (SEQ ID NO: 20) and an GPI anchor (SEQ ID NO:17) to mimic its naturally membrane nearby distribution.

### Neuronal Death Assessment

Neurons were fixed in 4% paraformaldehyde with 4% sucrose in PBS for 15 min followed with Hoechst staining. For neuronal death analysis, images are obtained using a Leica DMIRBE inverted microscope equipped with a 10× objective (NA 0.3) and a SPOT Insight 14-bit CCD camera with VisiView imaging software (Visitron Systems). Dead neurons were defined as condensed, round and shrinking nuclei from Hoechst staining. Cell death is analyzed by Cell Profiler and Cell Analyzer software.

### Quantification and Statistics

All statistics work was performed by Prism (GraphPad). All plotted data represent Mean t s.d.. Two-Way ANOVA analysis were used for statistical analyses unless otherwise indicated.

### Reagents

Following reagents were used in this study MK-801 maleate (BN338, Biotrend). DL-APV (BN0858, Biotrend), NMDA (BN0385, Biotrend).

### Example 2: Generation and Testing of Deletion Mutants of GluN2A and GluN2B

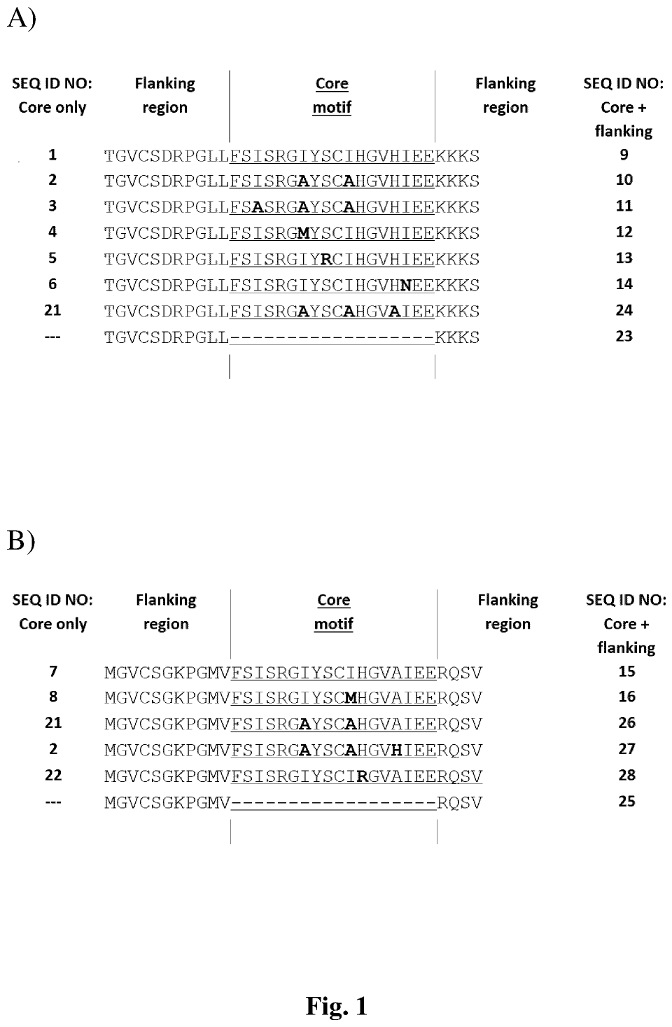

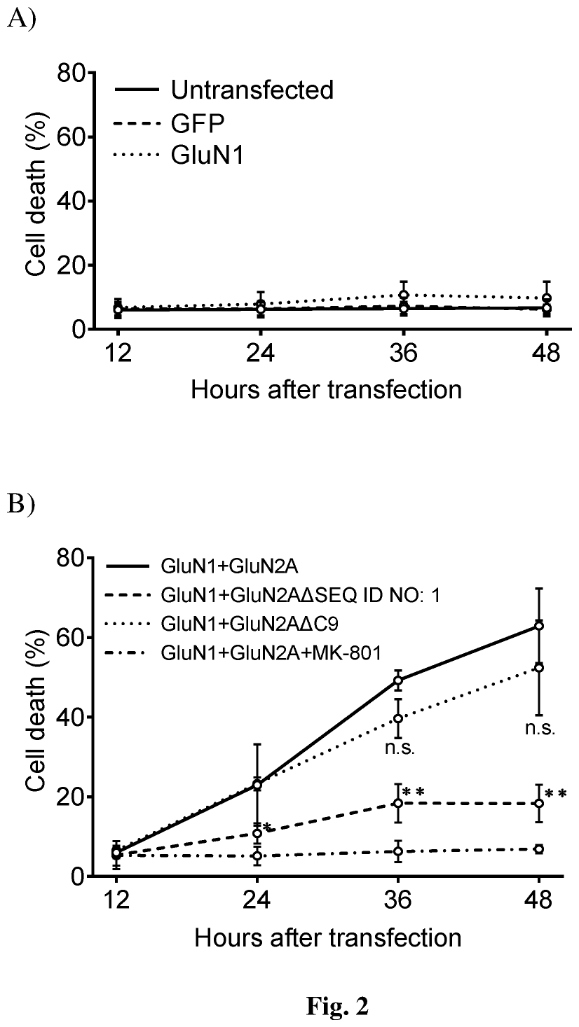

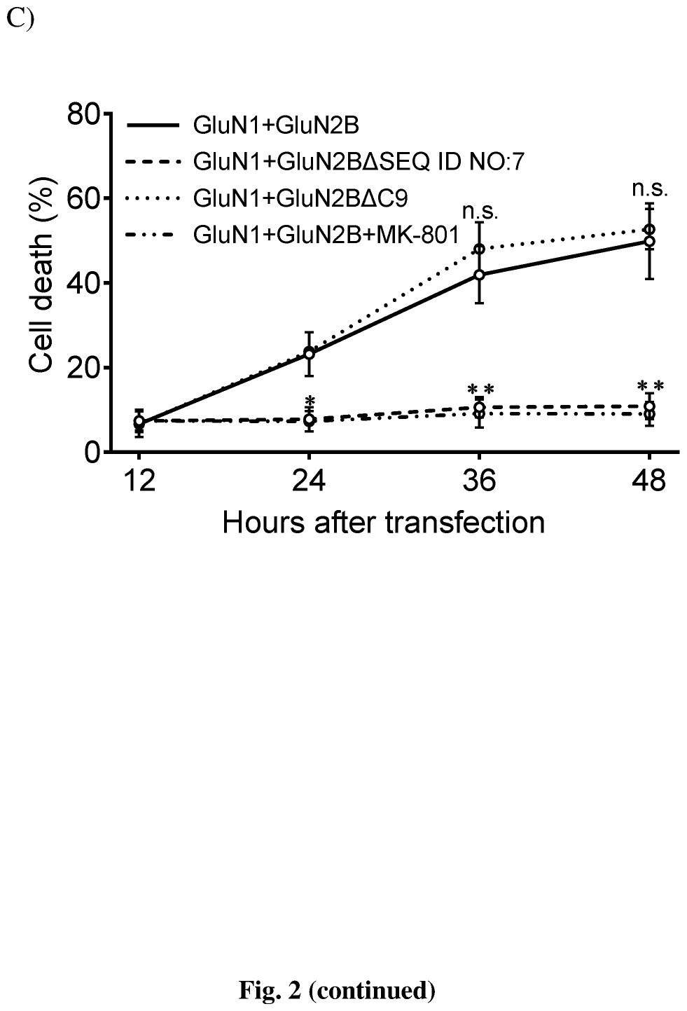

In order to investigate the role of GluN2A and GluN2B in NMDA receptor mediated toxicity, the inventors have built constructs and transfected the plasmids into HEK293 cells. Luminescent Cytotoxicity Assay were carried out 48 h after transfection, and cell death (%) is shown in FIG. 2.

As a result, neither GFP nor GluN1 alone (both serving as controls) could induce cell death. Combined expression of wild type rat GluN1 (UniProtKB-P35439; NMDZ1_RAT; sequence version of 1 Jun. 1994) and GluN2A (UniProtKB-Q00959; NMDE1_RAT, sequence version 2 of 15 Dec. 1998), the latter comprising the canonical sequence of SEQ ID NO:1 and also the sequence according to SEQ ID NO:9 including the flanking sequences), induced cell death in HEK293 cells, just as combined expression of GluN1 and a GluN2A mutant lacking the last 9 C-terminal amino acids of rat GluN2A did. However, combined expression of GluN1 and a rat GluN2A mutant (SEQ ID NO:41) lacking only the inventive sequence motif of SEQ ID NO:1 leads to a much lower level of cell death. Combined expression of GluN1 and rat GluN2A in presence of the non-competitive, general NMDA receptor inhibitor, MK-801, did not induce cell death (control experiment). A similar result was obtained for GluN2B. Combined expression of wild type rat GluN1 and GluN2B (UniProtKB-Q00960; NMDE2_RAT, sequence version 1 of Jun 1994)), the latter comprising the canonical sequence of SEQ ID NO:7 and flanking sequences, see SEQ ID NO:15), induced cell death in HEK293 cells, just as a deletion mutant lacking the last 9 C-terminal amino acids of rat GluN2B did. Combined expression of GluN1 and a GluN2B mutant (SEQ ID NO:42) lacking the inventive sequence motif of SEQ ID NO:7 did not lead to induction of cell death. Combined expression of GluN1 and GluN2B in presence of the non-competitive, general NMDA receptor inhibitor, MK-801, did also not induce cell death.

### Example 3: Generation and Testing of Mutants of the Inventive Sequence Motif in GluN2A and GluN2B

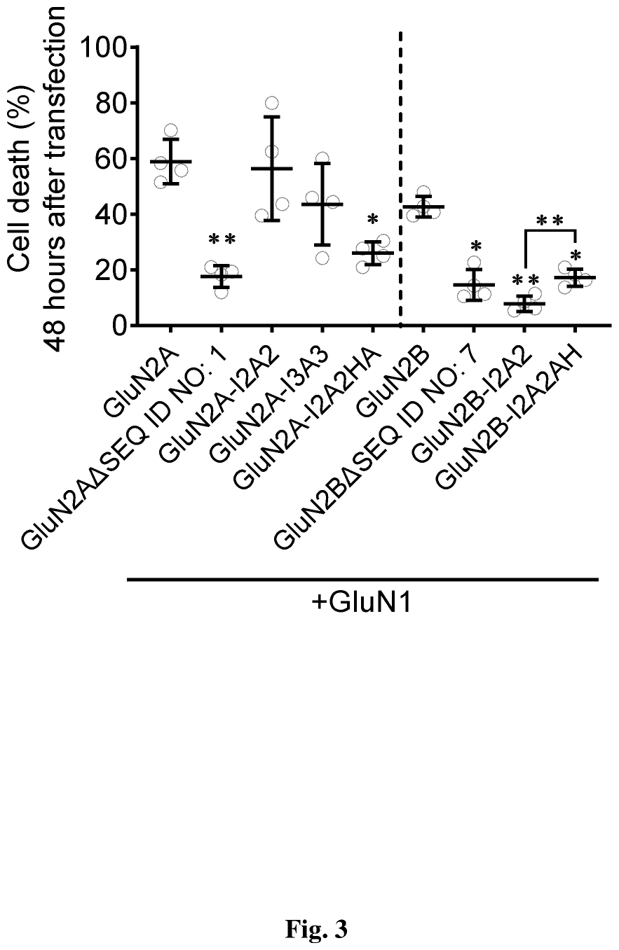

In a next step, the inventors tried to assess the importance of the isoleucine and other amino acid residues in the inventive sequence motif. The inventors have built constructs containing above-mentioned mutants and transfected the plasmids into HEK293 cells. Luminescent Cytotoxicity Assay were carried out 48 h after transfection, and cell death (%) is shown in FIG. 3.

As a result, the deletion mutants (GluN2A: lacking SEQ ID NO:1;GluN2B: lacking SEQ ID NO:7) substantially lost their capacity to induce cell death in HEK293 cells. Variants of GluN2A with mutations in the inventive sequence motif (see SEQ ID NO:2, SEQ ID NO:3 and SEQ ID NO:21) exhibited at least reduced levels of toxicity. For GluN2B, similar mutations essentially abrogated any toxic effect (see SEQ ID NO:21, and SEQ ID NO:2).

### Example 4: Assessment of the Impact of Further GluN2A and GluN2B Variants on Susceptibility Towards NMDA Receptor Mediated Toxicity

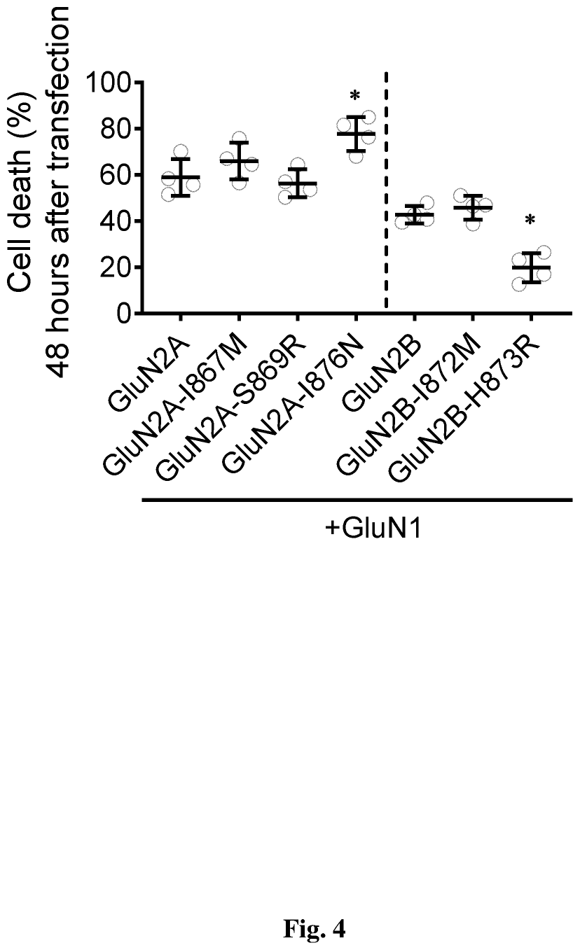

In view of the results obtained for the GluN2A and GluN2B mutants of the inventive sequence motif in example 3, the inventors further tested the impact of naturally occurring variations in said sequence motif in the human gene pool on susceptibility towards NMDA receptor mediated toxicity. The inventors have built constructs containing above-mentioned mutants selected from the SNP database from NCBI (for GRIN2A, https://www.ncbi.nlm.nih.gov/projects/SNP/snp_ref.cgi?showRare=on&chooseRs=coding&g o=Go&locusId=2903, and for GRIN2B, https://www.ncbi.nlm.nih.gov/projects/SNP/snp_ref.cgi?showRare=on&chooseRs=coding&g o=Go&locusId=2904) and transfected the plasmids into HEK293 cells. Luminescent Cytotoxicity Assay were carried out 48 h after transfection, and cell death (%) is shown in FIG. 4.

As a result, the naturally occurring variant sequences exhibiting the variant sequences can have a significant impact on susceptibility towards cell death from GluN2A and GluN2B proteins. For example, subjects exhibiting the sequence according to SEQ ID NO:6 instead of SEQ ID NO:1 in their GluN2A protein may have a significantly increased susceptibility towards NMDA mediated cell death. And on the contrary, subjects exhibiting SEQ ID NO:8 in their GluN2B protein instead of SEQ ID NO:7 may have a significantly decreased susceptibility towards NMDA mediated cell death.

### Example 5: Fusion Proteins Comprising a Peptide According to SEO ID NO:1 or According to SEQ ID NO:7 is Sufficient to Induce NMDA Receptor Mediated Toxicity

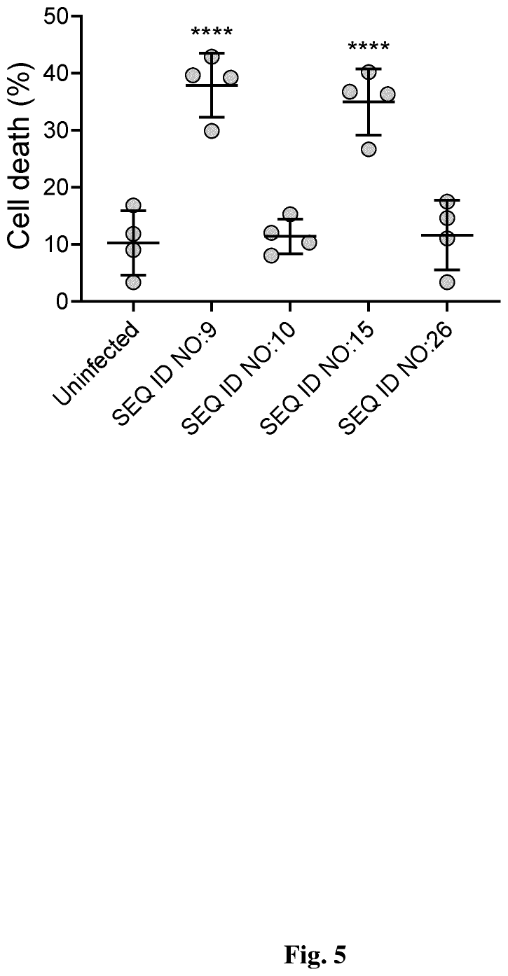

In a next step, the inventors assessed whether the inventive sequence motif on its own is sufficient to induce NMDA receptor mediated toxicity. For this purpose, the inventive core region of GluN2A (SEQ ID NO:1) and GluN2B (SEQ ID NO:7) plus flanking regions were expressed in mouse hippocampal neurons by way of infection with recombinant adeno-associated viruses. For this purpose, primary mouse hippocampal neurons were infected on DIV3 with the either rAAV-SEQ ID NO:9 (i.e., comprising SEQ ID NO:1), rAAV-SEQ ID NO: 10 (i.e., comprising SEQ ID NO:2), rAAV-SEQ ID NO:15 (i.e., comprising SEQ ID NO:7), and rAAV-SEQ ID NO:26 (i.e., comprising SEQ ID NO:3). Neurons were fixed on DIV17, stained with Hoechst and cell death was quantified. The results show that peptides comprising SEQ ID NO:1 and SEQ ID NO:7 are sufficient to induce cell death and that mutation of two of the alanine residues in the core motifs of GluN2A and GluN2B greatly reduces the cell death levels to approx. the levels of untransfected control (see FIG. 5). This experiment therefore shows, that the results obtained with full length GluN2A and GluN2B receptors can be reproduced by using only the critical core motif, which is therefore considered as being sufficient on its own to induce NMDA receptor mediated toxicity.

### Example 6: Example for a Diagnostic Kit According to the Present Invention

A kit according to the present invention may essentially be configured in a similar fashion as a standard kit for DNA paternity testing. The kit can contain a genomic DNA extraction kit and PCR/sequencing kit. The genomic DNA extraction kit contains the following: Lysis/Binding Buffer, Digestion Buffer, Wash Buffer 1, Wash Buffer 2, Elution Buffer, RNase A, Proteinase K, Spin columns for genomic DNA binding with collection tubes and additional collection tubes. The PCR/sequencing kit contains a 2× Mastermix for standard PCR, and primers for PCR and sequencing. Targets regions for human GRIN2A are amplified in two regions by the following primers:

Region 1 (Forward: GGGATCTGCCACAACGAGAA;
SEQ ID NO: 29; Reverse: CTCCCGTTTCCTTTCTGCCT;
SEQ ID NO: 30)
Region 1 could be sequenced with the following
primer (SEQ primer1: GCGTATTCTACATGCTGG;
SEQ ID NO: 33).
Region 2 (Forward: CCTATGCTTTGCAACTTGTCTCA;
SEQ ID NO: 31; Reverse: TTGGATGAAGTCAGCAGCTCTT;
SEQ ID NO: 32)
Region 2 could be sequenced with the following
primer (SEQ primer2: CAGGGCTCCTGCAAGAAG;
SEQ ID NO: 34).

Targets regions for SNP detection for human GRIN2B are amplified in two regions by the following primers

Region 1 (Forward: GAAGCTCTCTGGCTCACTGG;
SEQ ID NO: 35; Reverse: AACTGCCCAAATCCCACACA;
SEQ ID NO: 36)
Region 1 could be sequenced with the following
primer (SEQ primer3: CACAATGAGAAGAATGAGG;
SEQ ID NO: 39).
Region 2 (Forward: ACCTCAGCTCACCACATGAC;
SEQ ID NO: 37; Reverse: GATGACTCCCGTCGGATGAA;
SEQ ID NO: 38)
Region 2 could be sequenced with the following
primer (SEQ primer2: GGTATAAATTAGGCAAACT;
SEQ ID NO: 40).

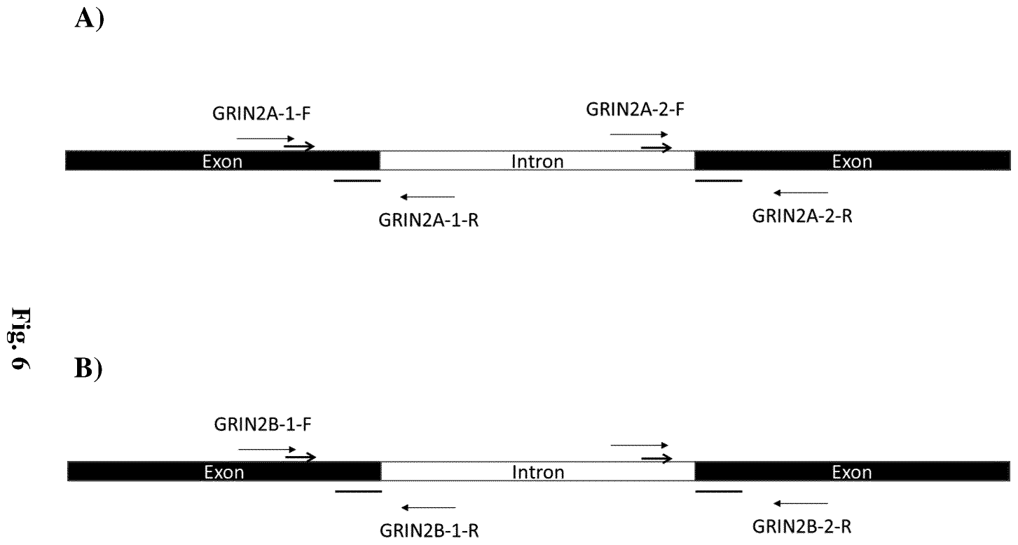

The samples on which the DNA extraction kit is used are taken from human subjects and could be a blood sample or a mouth swap. The sequencing concept is illustrated in FIG. 6.

1. A polypeptide comprising an amino acid sequence selected from the group consisting of: SEQ ID NO:1, SEQ ID NO:2, SEQ ID NO:3, SEQ ID NO:4, SEQ ID NO:5, SEQ ID NO:6, SEQ ID NO:7 and SEQ ID NO:8, or comprising a variant sequence thereof, wherein said variant sequence exhibits at least 80% sequence identity with a sequence selected from the group consisting of: SEQ ID NO:1, SEQ ID NO:2, SEQ ID NO:3, SEQ ID NO:4, SEQ ID NO:5, SEQ ID NO:6, SEQ ID NO:7 and SEQ ID NO:8, and wherein the polypeptide is at most 125 amino acids long.

2. The polypeptide according to claim 1, wherein the polypeptide comprises a sequence selected from the group consisting of SEQ ID NO:9, SEQ ID NO:10, SEQ ID NO: 11, SEQ ID NO:12, SEQ ID NO:13, SEQ ID NO:14, SEQ ID NO:15 and SEQ ID NO:16.

3. A fusion protein comprising the polypeptide of claim 1 and at least one further amino acid sequence heterologous to the amino acid sequence according to claim 1, in particular wherein the heterologous polypeptide sequence is selected from one or more of the group consisting of: a sequence of a membrane anchoring polypeptide, a sequence of a protein transduction domain and a sequence of a tag.

4. A nucleic acid encoding a polypeptide according to claim 1.

5. A method of treating cancer in a subject, in particular for treating neuroblastoma, comprising administering to said subject a polypeptide according to claim 1.

6. A cell comprising:
a) a polypeptide according to claim 1
b) a recombinantly expressed GluN2A protein, wherein the GluN2A protein comprises instead of the canonical sequence motif of SEQ ID NO:1 a variant sequence thereof, or wherein said aforementioned sequence motif of SEQ ID NO:1 is deleted; wherein the variant sequence leads to a GluN2A protein, which exhibits reduced toxic activity as compared to GluN2A protein comprising the canonical sequence motif of SEQ ID NO:1;
c) a recombinantly expressed GluN2B protein, wherein the GluN2B protein comprises instead of the canonical sequence motif of SEQ ID NO:7 a variant sequence thereof, or wherein said aforementioned sequence motif of SEQ ID NO:7 is deleted; wherein the variant sequence leads to a GluN2B protein, which exhibits reduced toxic activity as compared to GluN2B protein comprising the canonical sequence motif of SEQ ID NO:7.

7. The cell according to claim 6, wherein the variant sequence of SEQ ID NO:1 exhibits one or more of the following variant amino acid residues with respect to the sequence of SEQ ID NO:1: I3A, I7A, I11A, H15A; and/or wherein the variant sequence of SEQ ID NO:7 exhibits one or more of the following variant amino acid residues in the sequence of SEQ ID NO:7: I7A, I11A, H12R, A15H.

8. An antibody, nanobody or anticalin binding to the C-terminal cytoplasmic domain of a GluN2A or GluN2B protein, but not binding to a GluN2A or GluN2B deletion mutant lacking the region corresponding to amino SEQ ID NO:1 and SEQ ID NO:7, in particular wherein the antibody, nanobody or anticalin binds to sequence selected from the group consisting of: SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:21, SEQ ID NO:22, SEQ ID NO:24, SEQ ID NO:26, and SEQ ID NO:27.

9. The antibody, nanobody or anticalin of claim 8, wherein the antibody, nanobody or anticalin protects against, induces or ameliorates NMDA receptor mediated toxicity.

10. A method for studying NMDA receptor mediated toxicity comprising contacting a cell expressing an NMDA receptor with a polypeptide according to claim 1.

11. An ex vivo method of sequencing in a genomic nucleic acid sample of a subject a partial sequence of the GRIN2A gene (i.e., the gene encoding GluN2A protein) and/or a partial sequence of the GRIN2B gene (i.e., the gene encoding GluN2B protein), wherein the sequenced partial sequence of GRIN2A gene comprises at least the gene region usually encoding the canonical sequence of SEQ ID NO:1 protein, and wherein the sequenced partial sequence of GRIN2B gene comprises at least the gene region usually encoding the canonical sequence of SEQ ID NO:7.

12. A method for assessing the susceptibility of a subject to NMDA receptor mediated toxicity, the method comprising sequencing in a genomic nucleic acid sample of a subject a partial sequence of the GRIN2A gene (i.e., the gene encoding GluN2A protein) and/or a partial sequence of the GRIN2B gene (i.e., the gene encoding GluN2B protein), wherein the sequenced partial sequence of GRIN2A gene comprises at least the gene region usually encoding the canonical sequence of SEQ ID NO:1 in human GluN2A protein, and wherein the sequenced partial sequence of GRIN2B gene comprises at least the gene region usually encoding the canonical sequence of SEQ ID NO:7 of human GluN2B protein, and wherein a subject with:
a) a canonical GluN2A (SEQ ID NO:1) and/or a canonical GluN2B (SEQ ID NO:7) sequence exhibits a regular susceptibility to NMDA receptor mediated toxicity,
b) a GluN2A (I867M) mutation exhibits an increased susceptibility to NMDA receptor mediated toxicity,
c) a GluN2A (S869R) mutation exhibits a regular susceptibility to NMDA receptor mediated toxicity,
d) a GluN2A (I876N) mutation exhibits an increased susceptibility to NMDA receptor mediated toxicity,
e) a GluN2B (I872M) mutation exhibits a regular susceptibility to NMDA receptor mediated toxicity, and
f) a GluN2B (H₈₇₃R) mutation exhibits a decreased susceptibility to NMDA receptor mediated toxicity.

13. A method of assessing protein-protein interactions comprising contacting a candidate protein with the polypeptide of claim 1.

14. A method for identifying a compound potentially interacting with the C-terminal cytoplasmic domain of a GluN2A or GluN2B protein, wherein the method comprises:
i) computer-assisted virtual docking of a candidate compound to an amino acid sequence according to SEQ ID NO:1 or SEQ ID NO:7 or to a variant thereof, the variant being selected from GluN2A (I867M) (see also SEQ ID NO:4), GluN2A (S869R) (see also SEQ ID NO:5), GluN2A (I876N) (see also SEQ ID NO:6), GluN2B (I872M) (see also SEQ ID NO:8), and GluN2B (H873R) (see also SEQ ID NO:22), wherein said amino acid sequence is provided in a virtual 3-D structure of a polypeptide comprising said amino acid sequence, and
ii) determining the docking score and/or internal strain for docking the candidate compound virtually to the amino acid sequence according to SEQ ID NO:1 or SEQ ID NO:7 or its variant, and optionally
iii) contacting in vitro or in vivo an N-methyl-D-aspartate (NMDA) receptor comprising a GluN2A or GluN2B subunit, respectively, with the candidate compound to determine whether the candidate compound modulates the activity of said NMDA receptor or not.

15. A kit comprising means to determine in a genomic nucleic acid sample of a subject the sequence of a partial sequence of the GRIN2A gene and/or a partial sequence of the GRIN2B gene, wherein the sequenced partial sequence of GRIN2A gene comprises at least the gene region usually encoding the canonical sequence of SEQ ID NO:1 of GluN2A protein, and wherein the sequenced partial sequence of GRIN2B gene comprises the gene region usually encoding the canonical sequence of SEQ ID NO:7 of GluN2B protein, in particular wherein the kit comprises a primer selected from the group of consisting of: SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39 and SEQ ID NO:40.

16. A method of providing a cell with a nucleic acid providing more resistance towards NMDA receptor mediated toxicity, the method comprising:
i) replacing in said cell a GRIN2A and/or GRIN2B genomic sequence linked with a higher susceptibility towards NMDA receptor mediated toxicity with a GRIN2A and/or GRIN2B sequence linked with a lower susceptibility towards NMDA receptor mediated toxicity; or
ii) site-directed mRNA editing of a GRIN2A and/or GRIN2B mRNA transcript linked with a higher susceptibility towards NMDA receptor mediated toxicity in said cell into a GRIN2A and/or GRIN2B mRNA sequence linked with a lower susceptibility towards NMDA receptor mediated toxicity.
